(19) United States
(12) Patent Application Publication
(10) Pub. No.: US 2019/0183639 A1
(43) Pub. Date: Jun. 20, 2019

(54) Transcatheter Mitral Valve: Off-Center Valve Design

(71) Applicant: St. Jude Medical, Cardiology Division, Inc., St. Paul, MN (US)

(72) Inventor: Brandon Moore, Minneapolis, MN (US)

(73) Assignee: St. Jude Medical, Cardiology Division, Inc., St. Paul, MN (US)

(21) Appl. No.: 16/208,674

(22) Filed: Dec. 4, 2018

Related U.S. Application Data
(60) Provisional application No. 62/607,493, filed on Dec. 19, 2017.

(51) Int. Cl. A61F 2/24

(52) U.S. Cl. CPC A61F 2/2418; A61F 2230/0069; A61F 2220/0008; A61F 2/2409

(57) ABSTRACT

A collapsible and expandable prosthetic mitral valve includes a stent and a flange. The stent has an inflow end, an outflow end, and a first central longitudinal axis extending from the inflow end to the outflow end in an expanded condition of the prosthetic mitral valve. A valve assembly is disposed within the stent. The flange is formed of a braided mesh and has a body portion coupled to the stent and a flared portion adjacent the inflow end of the stent. A second central longitudinal axis extends through the flared portion in the expanded condition of the prosthetic mitral valve and is offset from the first central longitudinal axis.

### CROSS-REFERENCE TO RELATED APPLICATIONS

This application claims the benefit of the filing date of U.S. Provisional Patent Application No. 62/607,493, filed Dec. 19, 2017, the disclosure of which is hereby incorporated by reference herein.

### BACKGROUND OF THE DISCLOSURE

The present disclosure relates to prosthetic heart valves and, in particular, to collapsible prosthetic mitral valves.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed or crimped to reduce its circumferential size.

When a collapsed prosthetic valve has reached the desired implant site in the patient (e.g., at or near the annulus of the patient's heart valve that is to be replaced by the prosthetic valve), the prosthetic valve can be deployed or released from the delivery apparatus and re-expanded to full operating size. For balloon-expandable valves, this generally involves releasing the entire valve, assuring its proper location, and then expanding a balloon positioned within the valve stent. For self-expanding valves, on the other hand, the stent automatically expands as the sheath covering the valve is withdrawn.

Two challenges that arise when designing and implanting transcatheter mitral valves are left ventricular outflow tract (LVOT) obstruction and electrical conduction problems. Both problems may occur when a prosthetic mitral valve extends too far into the left ventricle and/or at too much of an angle with respect to the longitudinal axis of the native mitral valve annulus. LVOT obstruction may occur when the physical structure of the mitral valve is positioned in the path of blood flowing from the left ventricle to the aorta through the aortic valve. Electrical conduction problems may occur when a metallic stent frame of a prosthetic mitral valve physically contacts the septum wall separating the left and right ventricles. For these and other reasons, there is still room for improvement in the design and transcatheter implantation of prosthetic mitral valves.

### BRIEF SUMMARY

According to one aspect of the disclosure, a collapsible and expandable prosthetic mitral valve includes a stent having an inflow end, an outflow end, and a first central longitudinal axis extending from the inflow end to the outflow end in an expanded condition of the prosthetic mitral valve. A valve assembly is disposed within the stent. A flange is formed of a braided mesh and has a body portion coupled to the stent and a flared portion adjacent the inflow end of the stent. A second central longitudinal axis extends through the flared portion in the expanded condition of the prosthetic mitral valve. The first central longitudinal axis is offset from the second central longitudinal axis.

According to another aspect of the disclosure, a method of implanting a prosthetic mitral valve includes introducing a delivery device to a native mitral valve annulus while the prosthetic mitral valve is maintained in a collapsed condition by the delivery device. The prosthetic mitral valve is transitioned into an expanded condition so that a stent of the prosthetic mitral valve is positioned within the native mitral valve annulus to implant the prosthetic mitral valve. The stent includes a valve assembly disposed therein. Upon the transition, a flared portion of a flange of the prosthetic mitral valve contacts an atrial side of the native mitral valve annulus, the flange being formed of a braided mesh and having a body portion coupled to the stent. Upon implantation of the prosthetic mitral valve, the native mitral valve annulus has a first central longitudinal axis, and the stent has a second central longitudinal axis offset from the first central longitudinal axis.

### BRIEF DESCRIPTION OF THE DRAWINGS

FIG. 1 is a highly schematic cutaway representation of a human heart showing various delivery approaches.

FIG. 2 is a highly schematic representation of a native mitral valve and associated cardiac structures.

FIG. 3A is a side view of a prosthetic heart valve according to the prior art.

FIG. 3B is a highly schematic longitudinal cross-section of the prosthetic heart valve of FIG. 3A.

FIG. 4A is a side view of a prosthetic heart valve according to an aspect of the disclosure.

FIG. 4B is a side view of the prosthetic heart valve of FIG. 4A rotated about its longitudinal axis.

FIG. 4C is a highly schematic longitudinal cross-section of the prosthetic heart valve of FIG. 4A.

FIG. 4D is an enlarged, isolated perspective view of an anchor feature of the prosthetic heart valve of FIG. 4A.

FIG. 4E is a side view of the prosthetic heart valve of FIG. 4A in a stage of manufacture.

FIG. 4F is a highly schematic longitudinal cross-section of the prosthetic heart valve of FIG. 4A in a collapsed condition.

FIG. 4G is a highly schematic representation of the prosthetic heart valve of FIG. 4A implanted into a native mitral valve annulus.

FIG. 4H is a highly schematic bottom view of the outflow end of the prosthetic heart valve of FIG. 4A.

FIG. 4I is a highly schematic bottom view of the outflow end of a prosthetic heart valve according to another aspect of the disclosure.

FIG. 5A is a side view of a prosthetic heart valve according to a further aspect of the disclosure.

FIG. 5B is a side view of the prosthetic heart valve of FIG. 5A rotated about its longitudinal axis.

FIG. 5C is a highly schematic top view of the inflow end of the prosthetic heart valve of FIG. 5A.

FIG. 5D is a highly schematic bottom view of the outflow end of the prosthetic heart valve of FIG. 5A.

FIG. 5E is a highly schematic longitudinal cross-section of the prosthetic heart valve of FIG. 5A in the expanded condition.

FIG. 5F is a highly schematic longitudinal cross-section of the prosthetic heart valve of FIG. 5A in the collapsed condition.

FIG. 5G is a highly schematic representation of the prosthetic heart valve of FIG. 5A implanted into a native mitral valve annulus.

FIG. 5H is an enlarged, highly schematic cross-section of a portion of the flange of the prosthetic heart valve of FIG. 5A.

FIG. 6A is a side view of a prosthetic heart valve according to yet another aspect of the disclosure.

FIG. 6B is a side view of the prosthetic heart valve of FIG. 6A rotated about its longitudinal axis.

FIG. 6C is a bottom perspective view of the outflow end of the prosthetic heart valve of FIG. 6A.

FIG. 6D is a highly schematic top view of the inflow end of the prosthetic heart valve of FIG. 6A.

FIG. 6E is a highly schematic bottom view of the outflow end of the prosthetic heart valve of FIG. 6A.

FIG. 6F is a highly schematic longitudinal cross-section of the prosthetic heart valve of FIG. 6A in a collapsed condition.

FIG. 6G is a highly schematic representation of the prosthetic heart valve of FIG. 6A implanted into a native mitral valve annulus.

FIG. 7A is a highly schematic bottom view of the outflow end of a prosthetic heart valve according to another aspect of the disclosure.

FIG. 7B is a side view of a prosthetic heart valve according to a further aspect of the disclosure incorporating features of the prosthetic heart valve of FIG. 7A.

FIG. 7C is a side view of the prosthetic heart valve of FIG. 7B rotated about its longitudinal axis.

FIG. 7D is a highly schematic top view of the inflow end of the prosthetic heart valve of FIG. 7B.

FIG. 7E is a highly schematic bottom view of the outflow end of the prosthetic heart valve of FIG. 7B.

FIG. 7F is a highly schematic longitudinal cross-section of the prosthetic heart valve of FIG. 7B in the expanded condition.

FIG. 7G is a highly schematic longitudinal cross-section of the prosthetic heart valve of FIG. 7B in the collapsed condition.

FIG. 7H is a highly schematic representation of the prosthetic heart valve of FIG. 7B implanted into a native mitral valve annulus.

FIG. 8A is a side view of a prosthetic heart valve according to another aspect of the disclosure.

FIG. 8B is a highly schematic top view of the inflow end of the prosthetic heart valve of FIG. 8A.

FIG. 8C is a highly schematic representation of the prosthetic heart valve of FIG. 8A implanted into a native valve annulus.

FIG. 9A is a highly schematic top view of the inflow end of a prosthetic heart valve according to yet another aspect of the disclosure.

FIG. 9B is a highly schematic representation of the prosthetic heart valve of FIG. 9A implanted into a native valve annulus.

### DETAILED DESCRIPTION

Blood flows through the mitral valve from the left atrium to the left ventricle. As used herein, the term “inflow end,” when used in connection with a prosthetic mitral heart valve, refers to the end of the heart valve closest to the left atrium when the heart valve is properly implanted in a patient, whereas the term “outflow end,” when used in connection with a prosthetic mitral heart valve, refers to the end of the heart valve closest to the left ventricle when the heart valve is properly implanted in a patient. Also, as used herein, the terms “substantially,” “generally,” and “about” are intended to mean that slight deviations from absolute are included within the scope of the term so modified. Generally, materials described as being suitable for components in one embodiment may also be suitable for similar or identical components described in other embodiments.

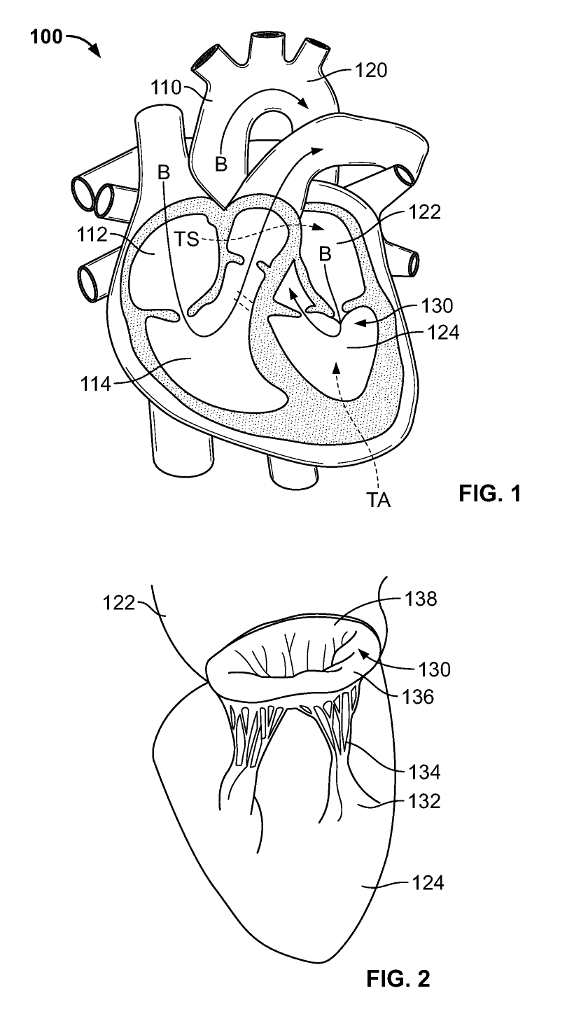

FIG. 1 is a highly schematic cutaway representation of human heart 100. The human heart includes two atria and two ventricles: right atrium 112 and left atrium 122, and right ventricle 114 and left ventricle 124. Heart 100 further includes aorta 110 and aortic arch 120. Disposed between left atrium 122 and left ventricle 124 is mitral valve 130. Mitral valve 130, also known as the bicuspid valve or left atrioventricular valve, is a dual-flap valve that opens as a result of increased pressure in left atrium 122 as it fills with blood. As atrial pressure increases above that of left ventricle 124, mitral valve 130 opens and blood passes into left ventricle 124. Blood flows through heart 100 in the direction shown by arrows “B”.

A dashed arrow, labeled “TA,” indicates a transapical approach of implanting a prosthetic heart valve, in this case to replace the mitral valve. In transapical delivery, a small incision is made between the ribs and into the apex of left ventricle 124 to deliver the prosthetic heart valve to the target site. A second dashed arrow, labeled “TS,” indicates a transseptal approach of implanting a prosthetic heart valve in which the valve is passed through the septum between right atrium 112 and left atrium 122. Other approaches for implanting a prosthetic heart valve are also possible.

FIG. 2 is a more detailed schematic representation of native mitral valve 130 and its associated structures. As previously noted, mitral valve 130 includes two flaps or leaflets, posterior leaflet 136 and anterior leaflet 138, disposed between left atrium 122 and left ventricle 124. Cord-like tendons, known as chordae tendineae 134, connect the two leaflets 136, 138 to the medial and lateral papillary muscles 132. During atrial systole, blood flows from higher pressure in left atrium 122 to lower pressure in left ventricle 124. When left ventricle 124 contracts in ventricular systole, the increased blood pressure in the chamber pushes leaflets 136, 138 to close, preventing the backflow of blood into left atrium 122. Since the blood pressure in left atrium 122 is much lower than that in left ventricle 124, leaflets 136, 138 attempt to evert to the low pressure regions. Chordae tendineae 134 prevent the eversion by becoming tense, thus pulling on leaflets 136, 138 and holding them in the closed position.

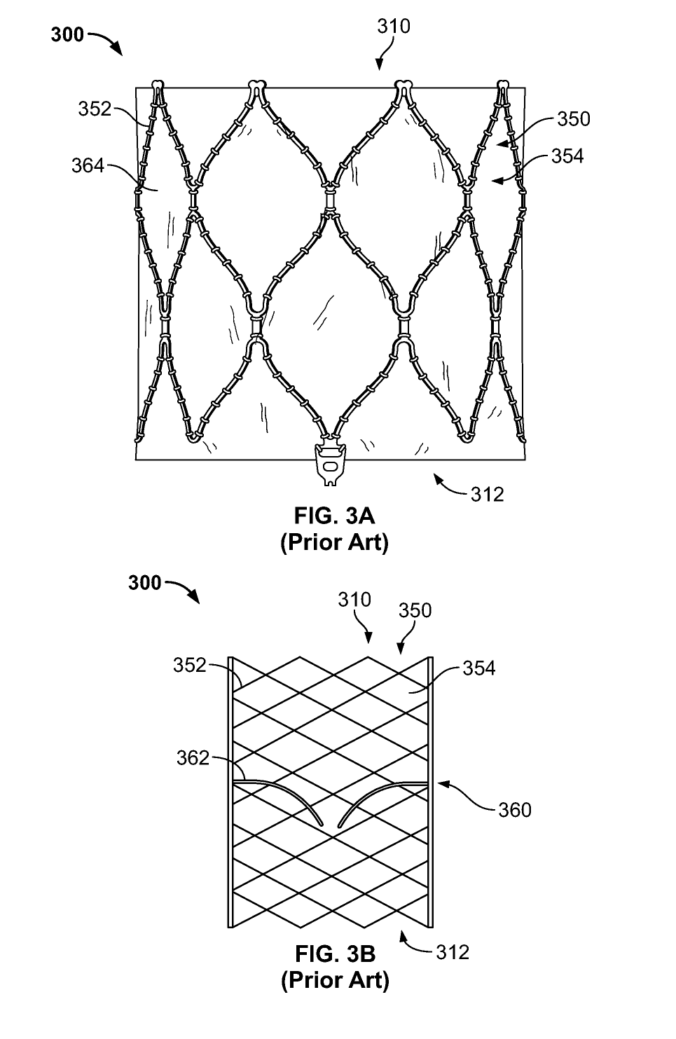

FIGS. 3A and 3B are a side view and a longitudinal cross-sectional view of prosthetic heart valve 300 according to the prior art. Prosthetic heart valve 300 is a collapsible prosthetic heart valve designed to replace the function of the native mitral valve of a patient (see native mitral valve 130 of FIGS. 1-2). Generally, prosthetic valve 300 has a substantially cylindrical shape with inflow end 310 and outflow end 312. When used to replace native mitral valve 130, prosthetic valve 300 may have a low profile so as not to interfere with atrial function in the native valve annulus.

Prosthetic heart valve 300 may include stent 350, which may be formed from biocompatible materials that are capable of self-expansion, such as, for example, shape-memory alloys including Nitinol. Stent 350 may include a plurality of struts 352 that form cells 354 connected to one another in one or more annular rows around the stent. Cells 354 may all be of substantially the same size around the perimeter and along the length of stent 350. Alternatively, cells 354 near inflow end 310 may be larger than the cells near outflow end 312. Stent 350 may be expandable to provide a radial force to assist with positioning and stabilizing prosthetic heart valve 300 in the native valve annulus.

Prosthetic heart valve 300 may also include a substantially cylindrical valve assembly 360 including a plurality of leaflets 362 (FIG. 3B) attached to a cuff 364 (FIG. 3A). Leaflets 362 replace the function of native mitral valve leaflets 136 and 138 described above with reference to FIG. 2. That is, leaflets 362 coapt with one another to function as a one-way valve. The valve assembly 360 of prosthetic heart valve 300 may include two or three leaflets, but it should be appreciated that prosthetic heart valve 300 may have more than three leaflets. Both cuff 364 and leaflets 362 may be wholly or partly formed of any suitable biological material, such as bovine or porcine pericardium, or polymers, such as polytetrafluoroethylene (PTFE), urethanes and the like. Valve assembly 360 may be secured to stent 350 by suturing to struts 352 or by using tissue glue, ultrasonic welding or other suitable methods.

When prosthetic heart valve 300 is implanted in a patient, for example at the annulus of native mitral valve 130, it is biased towards an expanded condition, providing radial force to anchor the valve in place. However, if the radial force is too high, damage may occur to heart tissue. If, instead, the radial force is too low, the heart valve may move from its implanted position, for example, into either left ventricle 124 or left atrium 122, requiring emergency surgery to remove the displaced valve. The potential for such movement may be heightened in mitral valve applications, particularly if a low profile valve is used.

Another potential issue with prosthetic heart valves is inadequate sealing between the prosthetic valve and the native tissue. For example, if prosthetic heart valve 300 is implanted at the annulus of mitral valve 130 in a patient, improper or inadequate sealing may result in blood flowing from left ventricle 124 into left atrium 122, even if leaflets 362 of valve assembly 360 are working properly. This may occur, for example, if blood flows in a retrograde fashion between the outer perimeter of prosthetic heart valve 300 and the native tissue at the site of implantation. This phenomenon is known as perivalvular (or paravalvular) leak (“PV leak”).

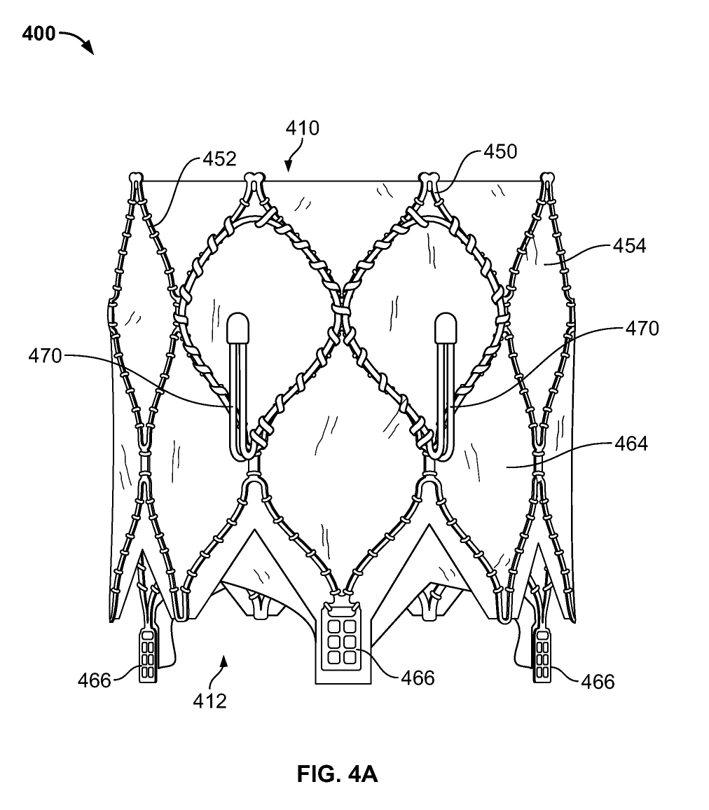

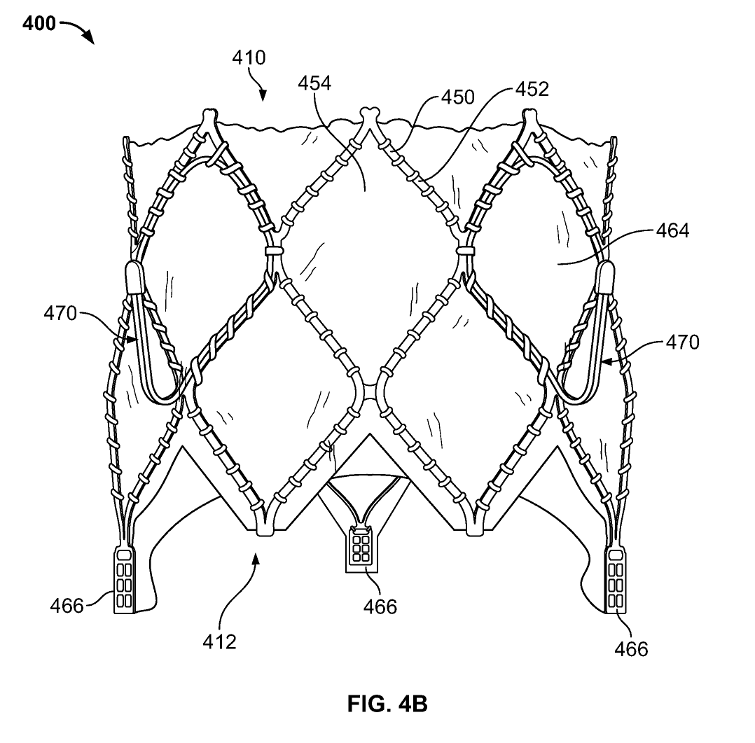

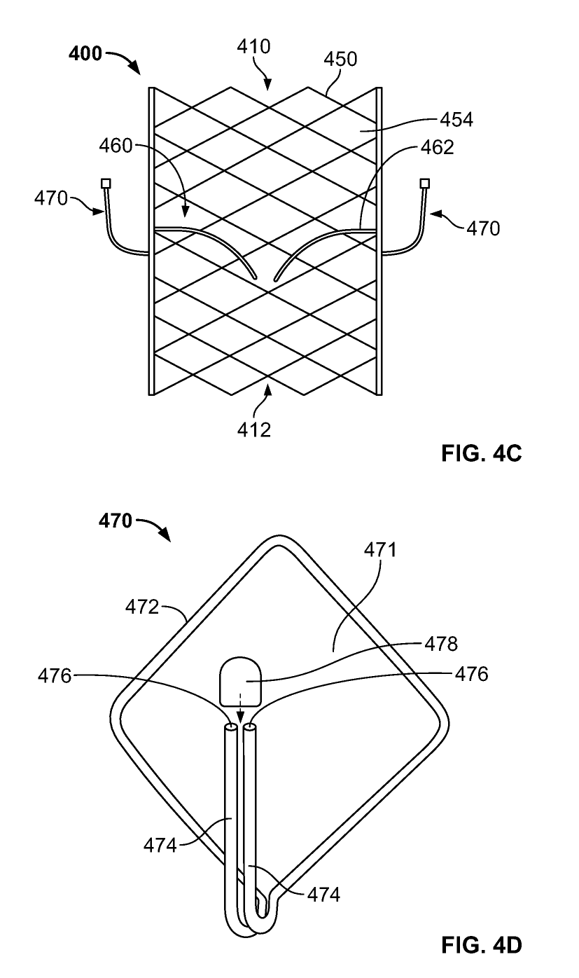

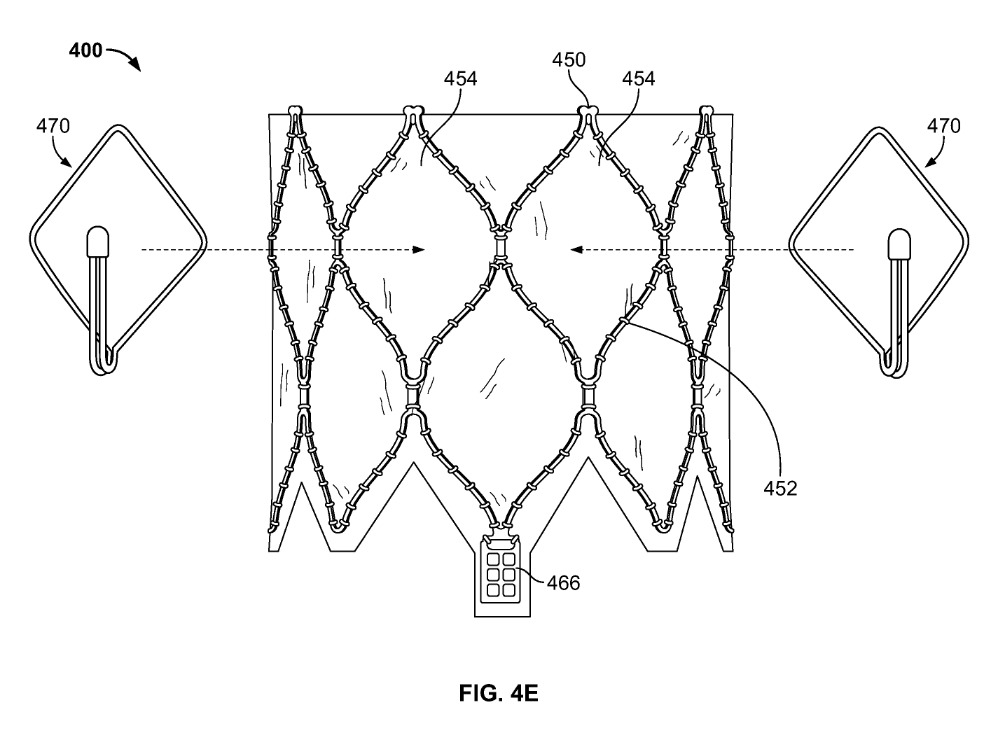

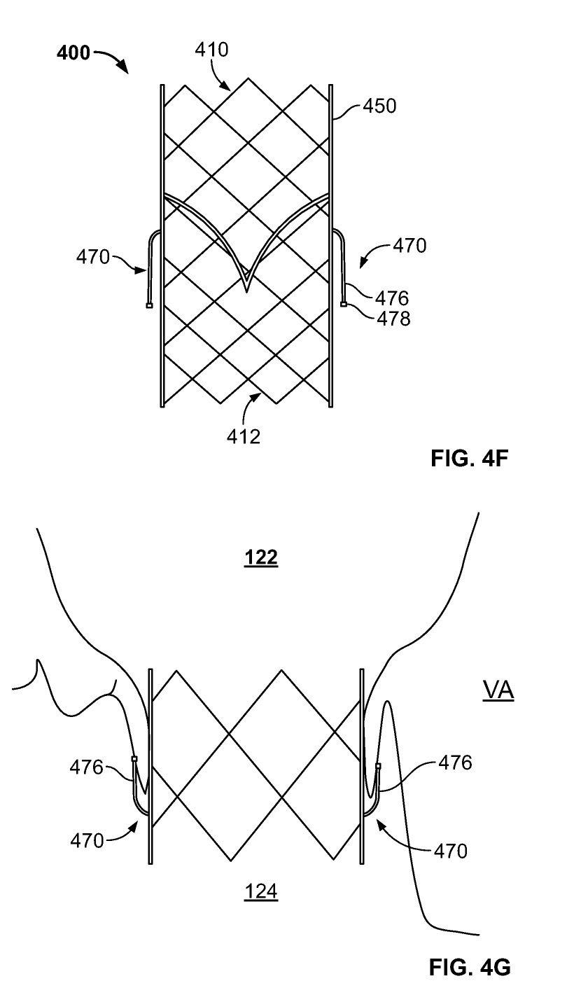

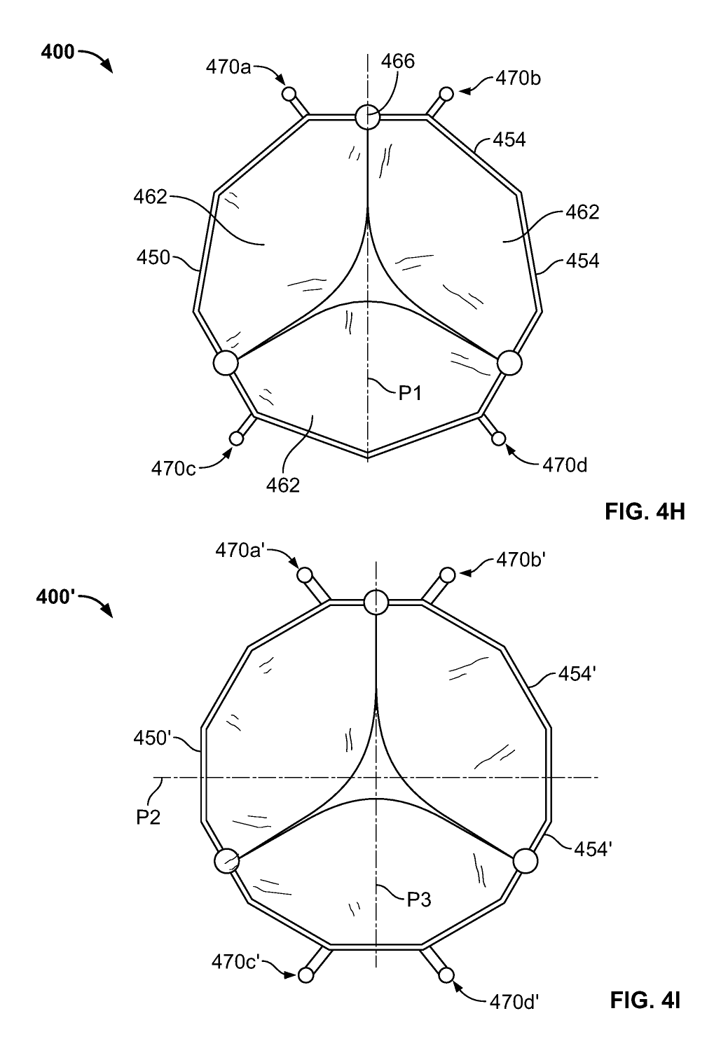

FIG. 4A is a side view of a prosthetic heart valve 400 in accordance with one embodiment of the disclosure. FIG. 4B shows prosthetic heart valve 400 rotated approximately 180 degrees about its longitudinal axis compared to FIG. 4A. Prosthetic heart valve 400 may be similar or identical to prosthetic heart valve 300 in certain respects. For example, prosthetic heart valve 400 is collapsible and expandable and designed for replacement of a native mitral valve, having a substantially cylindrical shape with an inflow end 410 and an outflow end 412. It should be understood that prosthetic heart valve 400 is not limited to replacement of mitral valves, and may be used to replace other heart valves. Prosthetic heart valve 400 may include stent 450, which may be similar to stent 350, having a plurality of struts 452 that form cells 454 connected to one another in one or more annular rows around stent 450. Stent 450 includes two annular rows of cells 454 of substantially similar size and shape, with nine cells in each row. However, it should be understood that a different number of rows of cells 454, as well as a different number of cells 454 per row, may be suitable. As illustrated, cells 454 are generally diamond shaped.

As discussed in relation to stent 350, stent 450 may be formed from a shape memory alloy, such as Nitinol. The struts 452 forming stent 450 may have a diameter of between about 0.020 inches (0.51 mm) and about 0.025 inches (0.64 mm), although other dimensions may be suitable. Forming stent 450 from struts 452 of a relatively large diameter may provide increased stiffness to stent 450, which may provide certain benefits, such as minimizing the deflection of commissure attachment features (CAFs) 466 during normal operation of prosthetic heart valve 400. On the other hand, forming stent 450 from struts 452 of a relatively small diameter may provide increased flexibility to stent 450, which may provide certain benefits, such as the capability to be collapsed to a smaller profile during delivery.

Prosthetic heart valve 400 may also include valve assembly 460 having three leaflets 462 attached to a cylindrical cuff 464. It should be understood that although native mitral valve 130 has two leaflets 136, 138, prosthetic heart valve 400 may have three leaflets, or more or fewer than three leaflets, provided that the leaflets act to allow one-way antegrade blood flow through the prosthetic heart valve 400. Because prosthetic heart valve 400 has three leaflets 462, it also has three CAFs 466, which provide points of attachment for adjacent leaflets 462 to stent 450. It should be understood that prosthetic heart valve 400 may alternatively include a pair of prosthetic leaflets and a corresponding pair of CAFs.

As with stent 350, stent 450 may be expandable to provide a radial force to assist with positioning and stabilizing prosthetic heart valve 400 in the native mitral valve annulus. However, prosthetic valve 400 includes additional securement features in the form of anchor arms 470 that hook under native mitral valve leaflets 136, 138 to help prevent prosthetic heart valve 400 from migrating into left atrium 122.

A single anchor arm 470 is shown in FIG. 4D. Anchor arm 470 may be formed of a single wire 472 bent or otherwise formed into a body portion 471 having a substantially diamond shape. Wire 472 is preferably formed of a shape-memory alloy such as Nitinol. In one example, wire 472 is formed of Nitinol having a diameter of about 0.015 inches (0.38 mm). As with struts 452 of stent 450, the diameter of wire 472 may be increased to provide increased stiffness or decreased to provide increased flexibility. Although the shape of body portion 471 may vary, it preferably corresponds to the geometry of a single cell 454 of stent 450. Wire 472 has two free end portions 474 that extend adjacent and substantially parallel to one another, and that are curved or hooked so as to lie at a spaced distance radially outward from body portion 471. Preferably, the tip 476 of each free end portion 474 is blunt and/or rounded to reduce the likelihood of tips 476 damaging the native tissue hooked by anchor arm 470. In addition or alternatively, a blunted and/or rounded end cap 478 may be assembled over or onto the tips 476 of free end portions 474 and fixed to tips 476, for example by welding, to provide an atraumatic tissue contact surface.

Prosthetic heart valve 400 is shown at a stage of manufacture in FIG. 4E to better illustrate the attachment of anchor arms 470 to prosthetic heart valve 400. After valve assembly 460 and cuff 464 have been attached to stent 450, anchor arms 470 may be coupled to prosthetic heart valve 400 at desired locations around stent 450. As shown in FIG. 4E, anchor arms 470 may be positioned within and/or adjacent to a selected cell 454 of stent 450 and connected to the prosthetic heart valve 400, for example by suturing the body portion 471 of anchor arm 470 to the struts 452 defining the perimeter of selected cell 454. The sutures coupling anchor arms 470 to prosthetic heart valve 400 may additionally pass through cuff 464. Forces applied to free end portions 474 are transmitted to the body portion 471 of anchor arm 470. With the above-described configuration of anchor arm 470 and its attachment to cell 454, those transmitted forces are distributed over a larger area of stent 450, providing better reinforcement than if free end portions 474 were sewn or otherwise directly connected to stent 450 without the use of body portion 471.

As noted above, wire 472 forming anchor arms 470 is preferably made from a shape-memory alloy. By using a shape-memory alloy, the shape of anchor arms 470 may be set, for example by heat setting, to take the illustrated shape in the absence of applied forces. However, forces may be applied to anchor arms 470 and to prosthetic heart valve 400 generally to reduce the radial size and/or bulk of the prosthetic heart valve when in the collapsed condition, which may facilitate intravascular (or other minimally invasive) delivery of the prosthetic heart valve via a delivery device (not shown). For example, as shown in FIG. 4F, prosthetic heart valve 400 may be transitioned to the collapsed condition, with free end portions 474 of anchor arms 470 distorted or “flipped” to point toward outflow end 412 rather than inflow end 410. Prosthetic heart valve 400 may be maintained in the collapsed condition, for example by a surrounding sheath of a delivery device (not shown), as prosthetic heart valve 400 is delivered to the site of native mitral valve 130. When in a desired position relative to native mitral valve 130, prosthetic heart valve 400 may be released from the delivery device. As constraining forces are removed from prosthetic heart valve 400, it begins to transition to the expanded condition, while anchor arms 470 move to their preset shape. Since anchor arms 470 are shape-set so that their free end portions 474 point toward inflow end 410, anchor arms 470 revert to that shape when released from the delivery device. As the free end portions 474 of anchor arms 470 transition from pointing toward outflow end 412 to pointing toward inflow end 410, native mitral valve leaflets 136, 138 are captured between the free end portions 474 and the body of stent 450, as shown in FIG. 4G. When hooked around native mitral valve leaflets 136, 138, anchor arms 470 help anchor prosthetic heart valve 400 within native valve annulus VA and are particularly effective at resisting migration of the prosthetic heart valve into left atrium 122. Distorting or flipping anchor arms 470 while prosthetic heart valve 400 is maintained in the collapsed condition may reduce the profile of the collapsed valve, although prosthetic heart valve 400 may alternatively be put in the collapsed condition without distorting or flipping anchor arms 470.

As described above, the stent 450 of prosthetic heart valve 400 may include two circumferential rows of annular cells 454, with each row containing nine cells 454. Although the use of nine cells 454 is merely an example, the use of an odd number of cells 454 in prosthetic heart valves for replacing native mitral valve 130 may cause difficulty in creating symmetry in the positioning of anchor arms 470 on the prosthetic heart valve. For example, it is preferable, although not necessary, to use two anchor arms 470 for each of the two native mitral valve leaflets to better distribute the forces caused by hooking or clamping native the mitral valve leaflets between anchor arms 470 and stent 450. With nine substantially equally-sized cells 454, or any other odd number of similarly sized cells, symmetry in the positioning of anchor arms 470 is difficult to achieve. FIG. 4H shows prosthetic heart valve 400 as viewed from outflow end 412. It should be understood that although stent 450 is illustrated as a regular nine-sided polygon (with each side representing a single cell 454), this representation is for purposes of clarity only and prosthetic heart valve 400, including stent 450, may take a substantially cylindrical shape when in the expanded condition. As shown in FIG. 4H, two anchor arms 470a and 470b may be coupled to stent 450 at adjacent cells 454, for example on cells 454 on either side of a CAF 466. The remaining two anchor arms 470c and 470d cannot be placed on adjacent cells 454 diametrically opposed to anchor arms 470a and 470b so as to maintain the symmetry of anchor arms 470. When positioning two pairs of anchor arms on substantially diametrically opposed portions of stent 450, it is preferable to maintain the symmetry of the anchor arms relative to at least one plane P1 dividing prosthetic heart valve 400. As shown in FIG. 4H, for a stent having nine substantially similarly-sized cells, this symmetry may be achieved by coupling the other pair of anchor arms 470c and 470d to stent 450 at two cells 454 that are separated by one cell 454. When implanting prosthetic heart valve 400, it is preferable to hook anchor arms 470a and 470b under the posterior leaflet 136 of native mitral valve 130, with anchor arms 470c and 470d hooked under the anterior leaflet 138 of native mitral valve 130. With this configuration, one CAF 466 abuts posterior leaflet 136 and two CAFs abut anterior leaflet 138.

The teachings provided above in connection with prosthetic heart valve 400 may be applied to a stent that is similar to stent 450, but that has an even number of cells. For example, FIG. 4I shows a prosthetic heart valve 400′ that incorporates a stent 450′ having two circumferential rows of twelve cells 454′ having substantially equal sizes. Similar to the illustration of FIG. 4H, stent 450′ in FIG. 4I is shown as a regular twelve-sided polygon for purposes of clarity only, and prosthetic heart valve 400′ and stent 450′ may be substantially cylindrical when in the expanded condition. The use of a stent 450′ having an even number of substantially similarly sized cells 454′ makes it easier to couple a first pair of anchor arms 470a′ and 470b′ to a first side of stent 450′ and a second pair of anchor arms 470c′ and 470d′ to a diametrically-opposed second side of stent 450′ while maintaining the symmetry of the anchor arms 470a′-470d′ relative to two planes P2, P3. In other words, the circumferential spacing between anchor arms 470a′ and 470b′ may be substantially equal to the spacing between anchor arms 470c′ and 470d′, while the circumferential spacing between anchor arms 470a′ and 470c′ may be substantially equal to the spacing between anchor arms 470b′ and 470d′. When prosthetic heart valve 400′ is implanted, this symmetry about two planes P2, P3 may provide for a more uniform distribution of forces than prosthetic heart valves exhibiting such symmetry in less than two planes (such as prosthetic heart valve 400 described above). In addition, the twelve-cell configuration may provide for more uniform expansion of the stent compared to the nine-cell configuration.

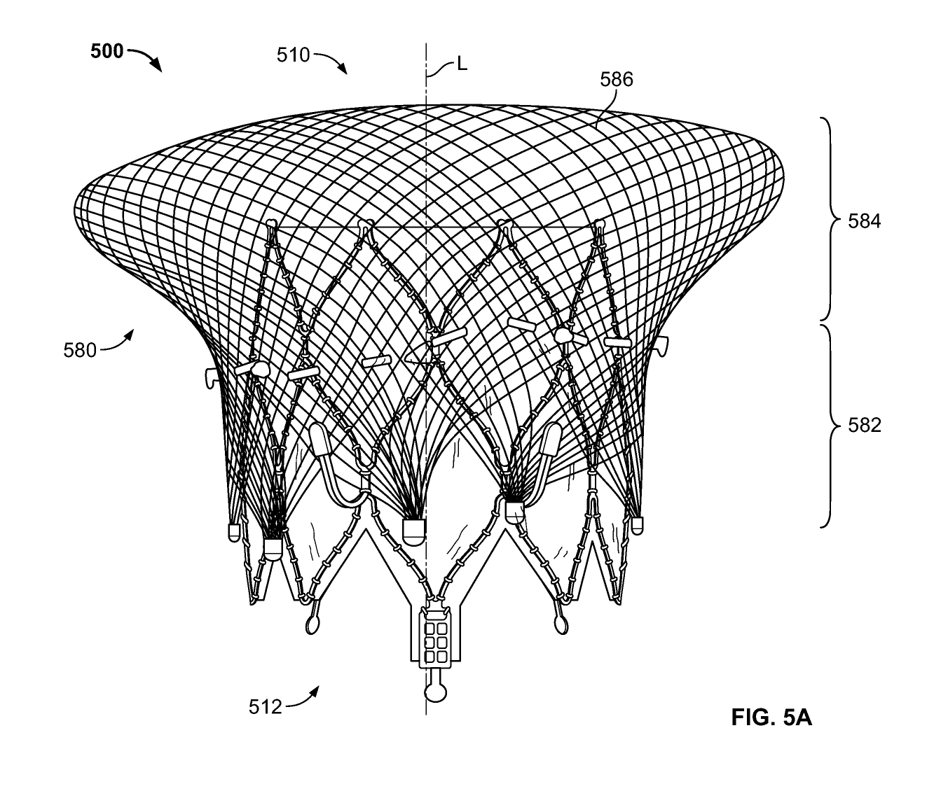

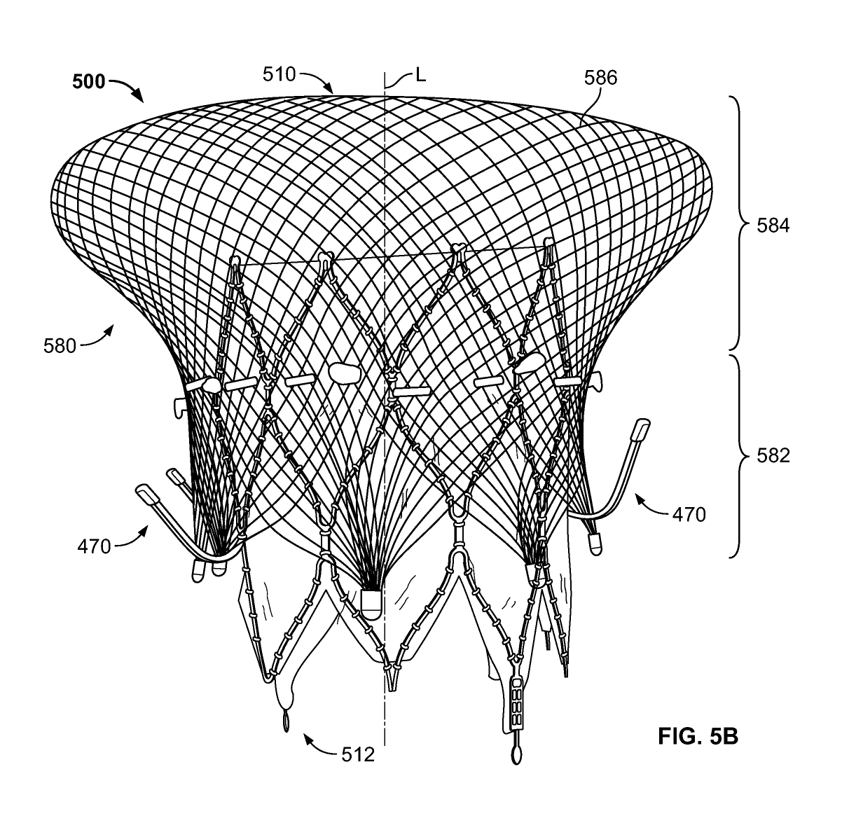

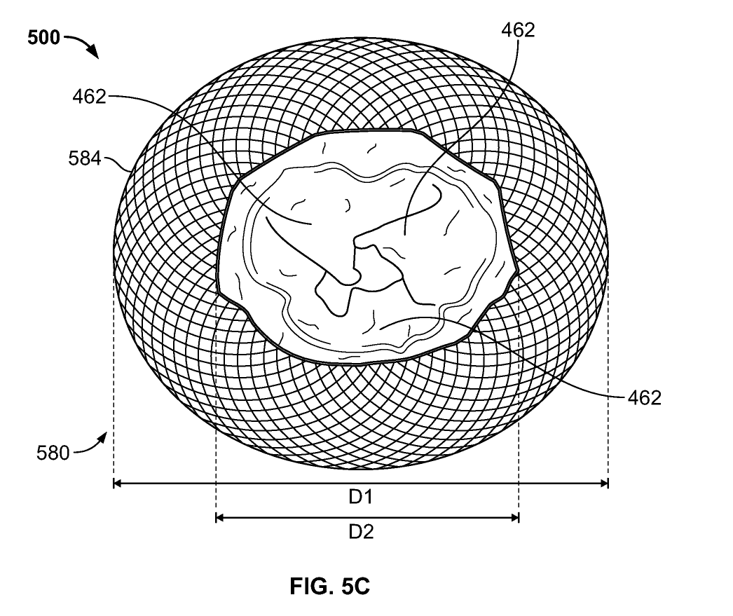

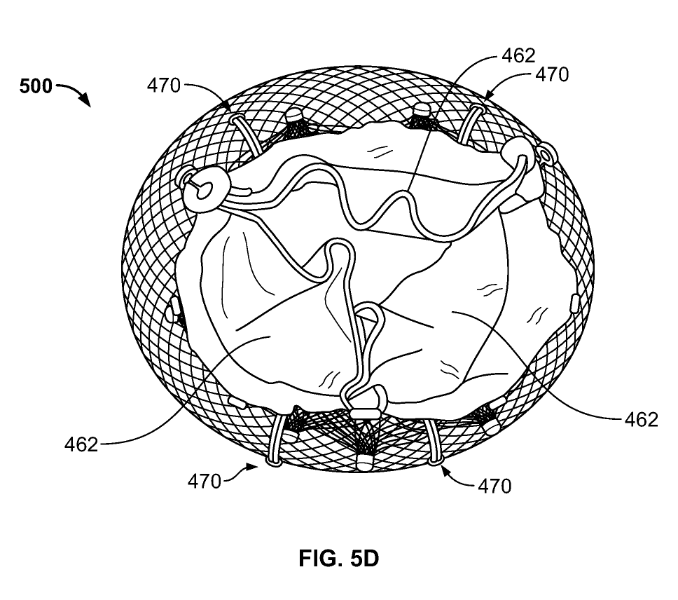

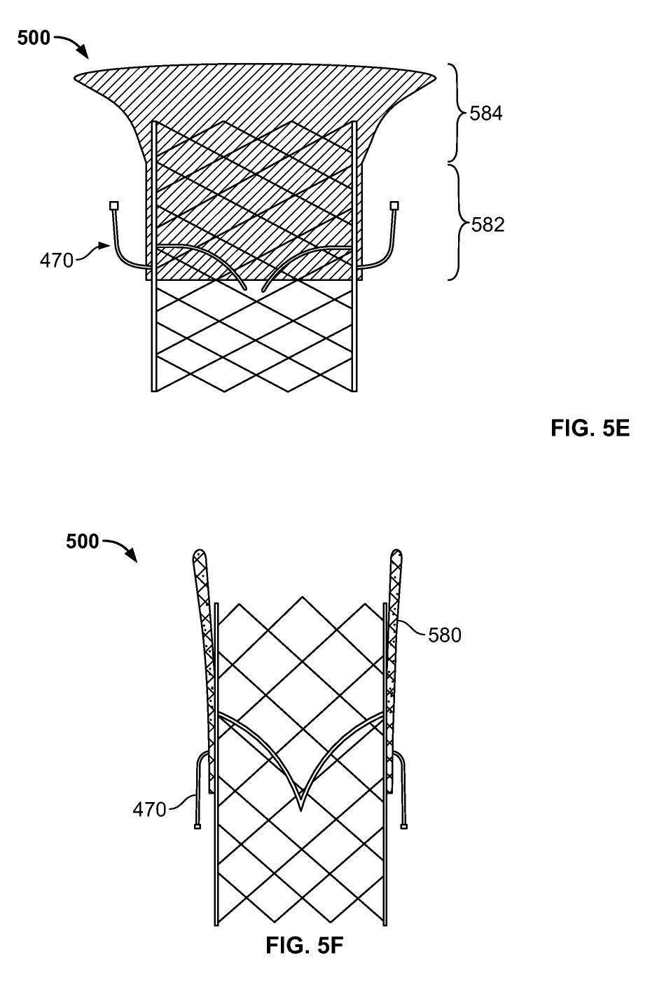

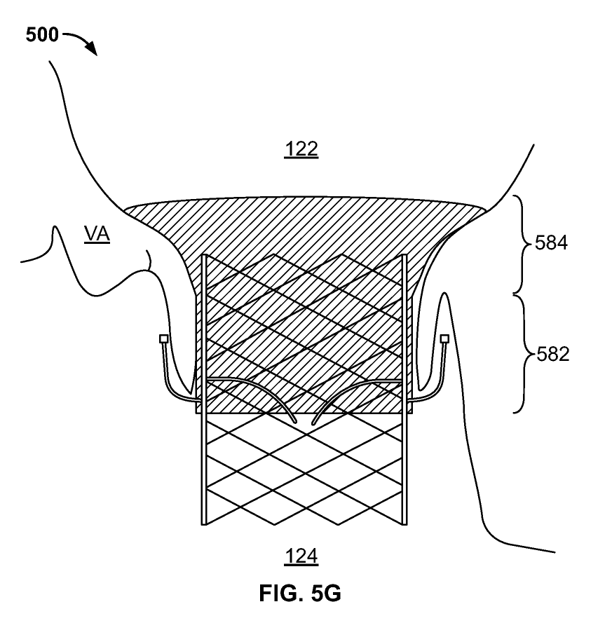

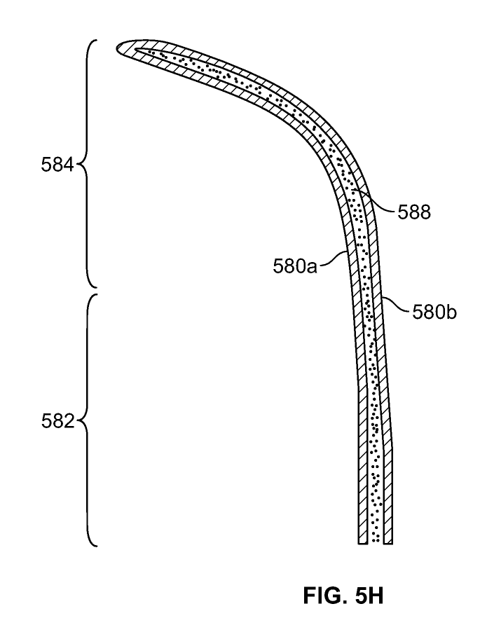

While prosthetic heart valve 400 may be used as shown and described above in connection with FIGS. 4A-I, a prosthetic heart valve may be provided with additional anchoring and/or sealing elements. For example, FIGS. 5A-D illustrate a prosthetic heart valve 500 that essentially comprises prosthetic heart valve 400 with a flange 580 coupled thereto. Flange 580 may facilitate the anchoring of heart valve 500 within native mitral valve annulus 130 and the prevention of PV leak. Flange 580 may be formed of a material braided to create various shapes and/or geometries to engage tissue. As shown in FIGS. 5A-D, flange 580 includes a plurality of braided strands or wires 586 arranged in three dimensional shapes. In one example, wires 586 form a braided metal fabric that is resilient, collapsible and capable of heat treatment to substantially set a desired shape. One class of materials which meets these qualifications is shape-memory alloys, such as Nitinol. Wires 586 may comprise various materials other than Nitinol that have elastic and/or memory properties, such as spring stainless steel, tradenamed alloys such as Elgiloy® and Hastelloy®, CoCrNi alloys (e.g., tradename Phynox), MP35N®, CoCrMo alloys, or a mixture of metal and polymer fibers. Depending on the individual material selected, the strand diameter, number of strands, and pitch may be altered to achieve the desired shape and properties of flange 580.

Flange 580 may include a body portion 582 terminating at an outflow end of the flange and a flared portion 584 terminating at an inflow end of the flange. Body portion 582 may be formed with a cylindrical or tubular geometry and may be configured to be circumferentially disposed around a portion of stent 450 and/or valve assembly 460. Flange 580 may be coupled to stent 450 (and optionally to valve assembly 460 and/or cuff 464) by sutures, for example. Flange 580 may be alternatively or additionally connected to stent 450 via ultrasonic welds, glue, adhesives, or other suitable means. When coupled to stent 450, body portion 582 of flange 580 is nearer outflow end 512 and flared portion 584 is nearer inflow end 510. When in the expanded condition, flared portion 584 extends a greater distance radially outwardly from the longitudinal axis L of prosthetic heart valve 500 than body portion 582. In other words, as shown in FIG. 5C, flared portion 584 may have a diameter D1 that is greater than the diameter D2 of body portion 582 when prosthetic heart valve 500 is in the expanded condition. In addition, the distance which flared portion 584 extends radially outwardly from longitudinal axis L may increase nearer inflow end 510.

Flange 580 may be preset to take the illustrated trumpet shape in the absence of external forces. As with stent 450 and anchor arms 470, flange 580 may be collapsed to a decreased profile to facilitate minimally invasive delivery. For example, prosthetic heart valve 500 may be transitioned from the expanded condition (FIGS. 5A-E) to the collapsed condition (FIG. 5F) and maintained in the collapsed condition by a surrounding sheath of a delivery device. Anchors 470 may flip and point toward outflow end 512 as described in connection with FIG. 4F, and flange 580 may collapse radially inwardly and become substantially cylindrical and/or significantly less flared than in the expanded condition. The body 582 of flange 580 may be positioned between anchor arms 470 and the remainder of stent 450. Prosthetic heart valve 500 may be delivered to the implant site in the collapsed condition and, when in the desired position relative to native mitral valve 130, may be transitioned to the expanded condition, for example by removing the surrounding sheath of the delivery device. During the transition from the collapsed condition to the expanded condition, anchor arms 470 revert to the preset shape as described in connection with FIG. 4F, capturing native mitral valve leaflets 136, 138 between anchor arms 470 and corresponding portions of stent 450. Flange 580 also transitions from the collapsed condition to the expanded condition, assuming its preset shape shown in FIG. 5G. When implanted and in the expanded condition, flange 580 provides a large surface area to help anchor prosthetic valve 500 within native valve annulus VA, and may be particularly effective at resisting movement of prosthetic heart valve 500 toward left ventricle 124. Specifically, flange 580 has an expanded diameter that is too large to pass through native valve annulus VA. Because flange 580 is coupled to stent 450, prosthetic heart valve 500 is restricted from migrating into left ventricle 124 during normal operation of prosthetic heart valve 500. Thus, the combination of anchor arms 470 engaged with the mitral valve leaflets, and flange 580 engaged with the tissue on the atrial side of the mitral valve annulus, helps to securely anchor prosthetic heart valve 500 within the mitral valve annulus and limits its migration toward either the left atrium or the left ventricle.

In addition to providing anchoring capabilities, flange 580 may improve sealing between prosthetic heart valve 500 and native valve annulus VA. In particular, as shown in FIG. 5H, flange 580 may be formed with an outer layer 580a and an inner layer 580b, for example by folding one portion of braided wires 586 over another portion of braided wires 586. A fabric layer 588, such as a polyester fabric, may be inserted or sandwiched between outer layer 580a and inner layer 580b. Fabric layer 588 may enhance tissue ingrowth into prosthetic heart valve 500 after implantation and may also enhance the fluid seal, and thus help prevent PV leak, between the outer diameter of prosthetic heart valve 500 and the adjacent portions of native mitral valve annulus VA. Although flange 580 is described as being folded over onto itself, alternative configurations may be suitable for holding fabric layer 588, for example by weaving or braiding two separate layers of braided wires 586 together. In a variation hereof, a single fabric layer 588 may be applied to the outside surface of flange 580, to the inside surface of flange 580, or to both the outside and inside surfaces of flange 580 to improve sealing between prosthetic heart valve 500 and native valve annulus VA.

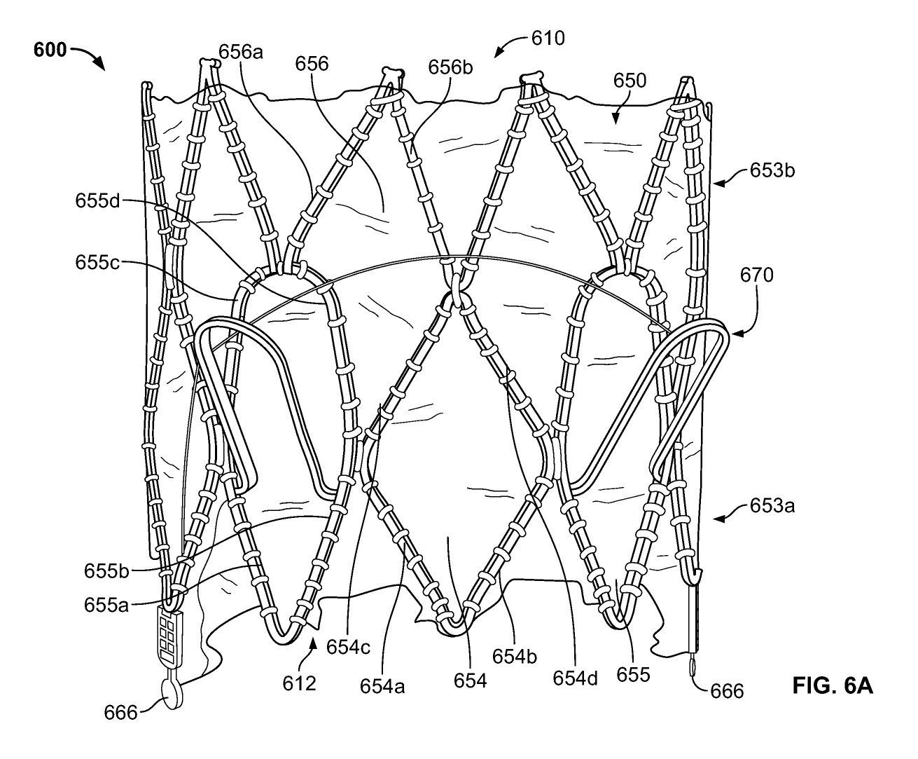

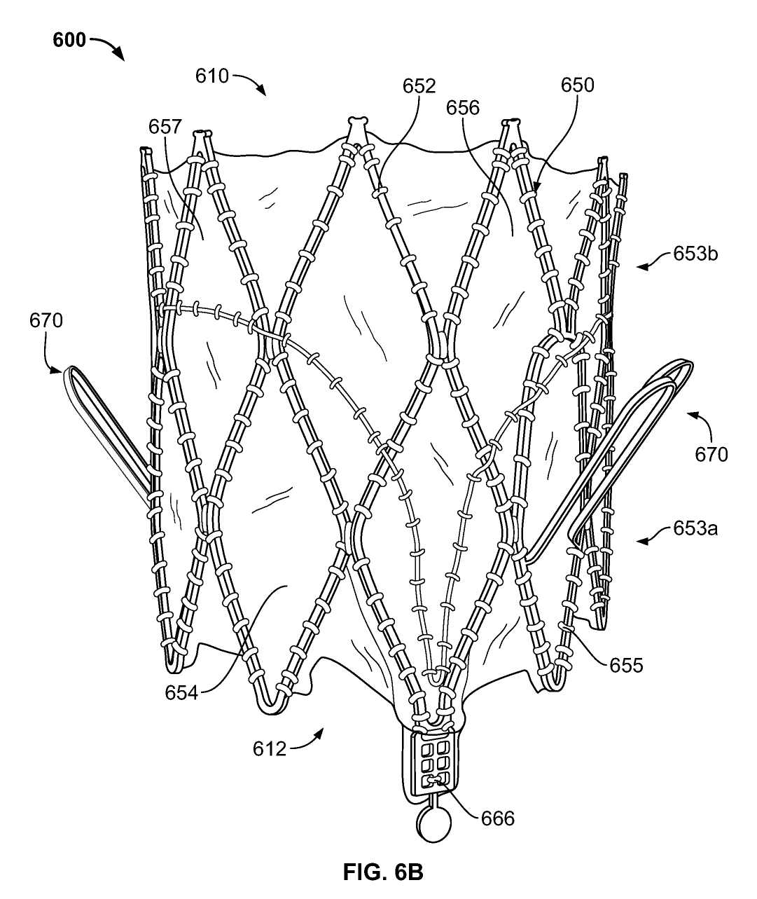

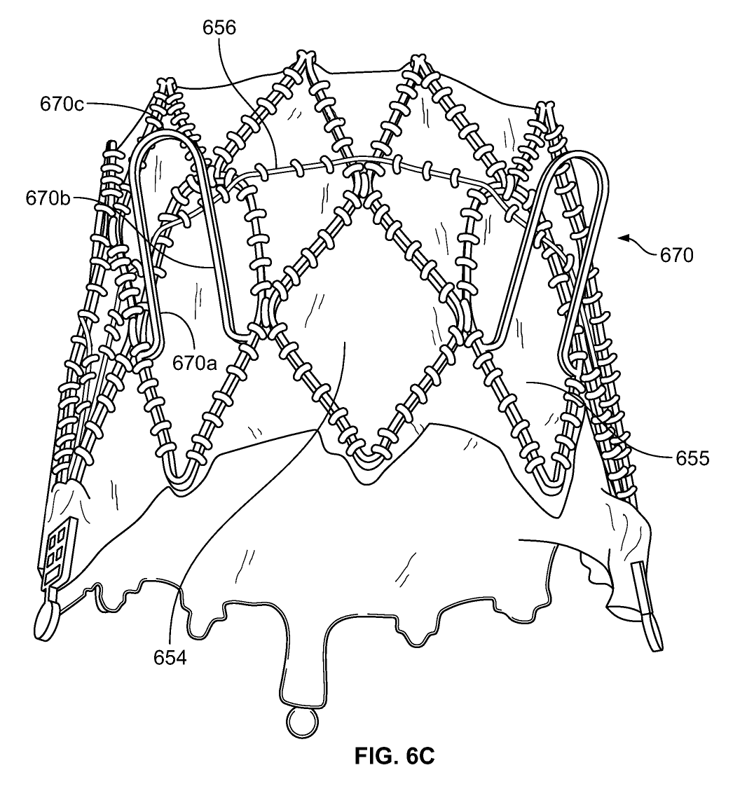

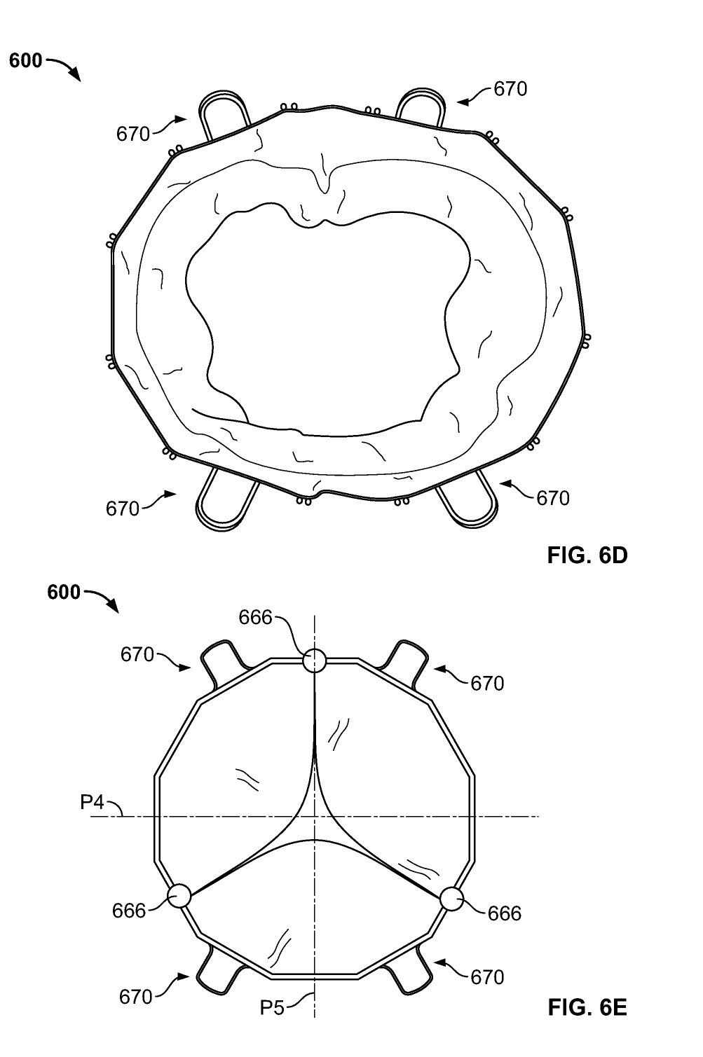

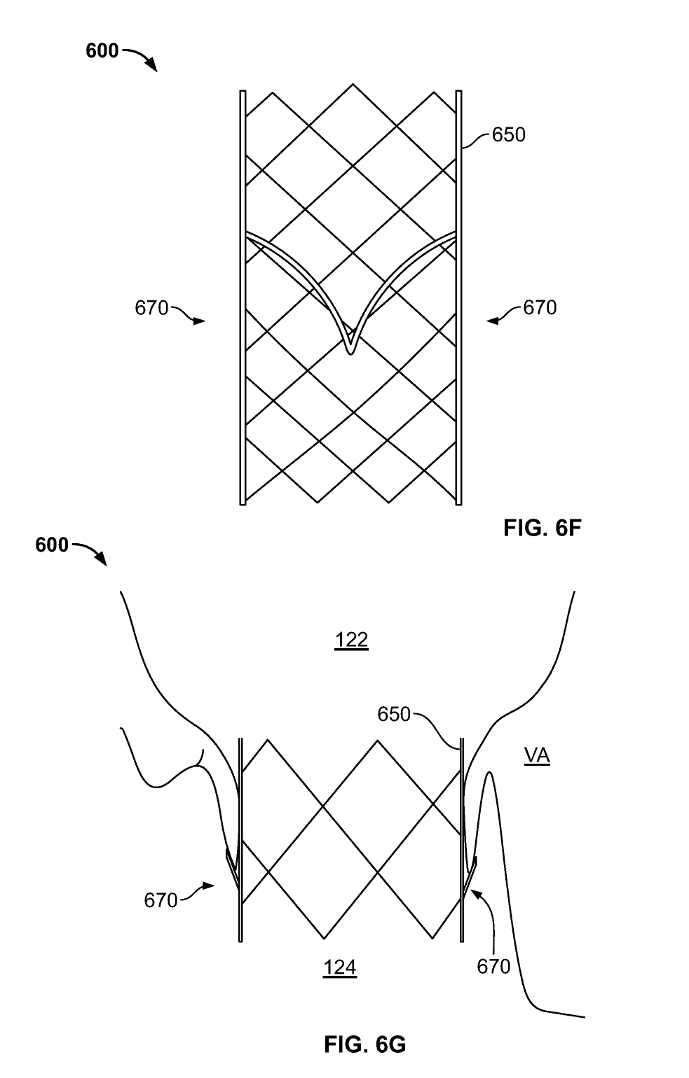

FIG. 6A is a side view of prosthetic heart valve 600 in accordance with a further embodiment of the disclosure. FIG. 6B illustrates prosthetic heart valve 600 rotated approximately 90 degrees about its longitudinal axis compared to FIG. 6A. Prosthetic heart valve 600 may be similar to prosthetic heart valve 300 in certain respects. For example, prosthetic heart valve 600 is collapsible and expandable and designed for replacement of a native mitral valve, having a substantially cylindrical shape with an inflow end 610 and an outflow end 612. Prosthetic heart valve 600 may also include a valve assembly having three leaflets attached to a cylindrical cuff, in substantially the same manner as described above in connection with prosthetic heart valve 400. It should be understood that prosthetic heart valve 600 is not limited to replacement of mitral valves, and may be used to replace other heart valves.

Prosthetic heart valve 600 may include stent 650, which generally extends between inflow end 610 and outflow end 612 and includes a plurality of struts 652 forming two circumferential rows of cells 653a, 653b. CAFs 666 may be included near outflow end 612. First row of cells 653a is disposed adjacent outflow end 612 and includes fully symmetric cells 654 alternating with second cells 655. Fully symmetric cells 654 may be substantially diamond-shaped and include four substantially straight struts 654a-d of equal length. Cells 654 are fully symmetric in that they are symmetric about a vertical line extending from the intersection of struts 654a and 654b to the intersection of struts 654c and 654c, and about a horizontal line extending from the intersection of struts 654a and 654c to the intersection of struts 654b and 654d. Cells 655 may include a pair of substantially straight struts 655a, 655b which form a V-shape attached to two substantially curved struts 655c, 655d. Cells 655 are partially symmetric in that they are symmetric only about a vertical line extending from the intersection of struts 655a and 655b to the intersection of struts 655c and 655d. Engaging arms 670 may be nested within each cell 655. Engaging arms 670 may be pivotably connected to cells 655 and configured to engage portions of heart tissue (e.g., native mitral valve leaflets) when prosthetic heart valve 600 is deployed in a patient, similar to anchor arms 470 described above. Second row of cells 653b may include a plurality of asymmetric cells 656 formed by two struts shared with cells from first row 653a (e.g., struts 654c and 655d or struts 654d and 655c) and two substantially straight struts 656a, 656b. Second row of cells 653b may also include a plurality of fully symmetric cells 657 substantially similar or identical to fully symmetric cells 654.

As shown in FIGS. 6A-E, stent 650 is formed of two rows of cells, each row having twelve cells and is thus referred to as a twelve-cell configuration. The considerations regarding the placement of engaging arms 670 around the circumference of stent 650 are similar to those described above with respect to the placement of anchor arms 470′ on twelve-cell stent 450′. In particular, first row of cells 653a may include two sets of three fully symmetric cells 654 on diametrically opposing portions of stent 650. Between each set of fully symmetric cells 654 may be another set of three cells, each set including two partially symmetric cells 655 having engaging arms 670 nested therein with a fully symmetric cell 654 positioned between the two partially symmetric cells 655. Because stent 650 has an even number of cells in first circumferential row 653a, in this case twelve, engaging arms 670 may be positioned symmetrically relative to two planes P4, P5, each bisecting prosthetic heart valve 600.

Each engaging arm 670 may be formed of a shape-memory alloy, and is preferably formed from the same material as stent 650. For example, stent 650 and engaging arms 670 may be formed from a single tube of Nitinol, for example by laser cutting. Engaging arms 670 may include two substantially parallel struts 670a, 670b connected to one another by rounded strut 670c. Engaging arms 670 may be shape set, for example by heat setting, so that in the absence of external forces, the free end of engaging arm 670 defined by strut 670c is positioned radially outwardly from the partially symmetric cell 655 in which the engaging arm is nested. However, forces may be applied to engaging arms 670 and to prosthetic heart valve 600 generally to reduce the radial size and/or bulk of the prosthetic heart valve when in the collapsed condition, which may facilitate intravascular (or other minimally invasive) delivery of the prosthetic heart valve via a delivery device (not shown).

For example, as shown in FIG. 6F, prosthetic heart valve 600 may be transitioned to the collapsed condition, with engaging arms 670 constrained so that each engaging arm is positioned substantially within a surface defined by the partially symmetric cell 655 in which the engaging arm is nested. In other words, when in the collapsed condition shown in FIG. 6F, engaging arms 670 do not protrude a significant distance radially outwardly from stent 650. Prosthetic heart valve 600 may be held in the collapsed condition by the delivery device as it is delivered to native mitral valve 130. When in a desired position relative to native mitral valve 130, prosthetic heart valve 600 may be released from the delivery device. As constraining forces are removed from prosthetic heart valve 600, it begins to transition to the expanded condition, while engaging arms 670 move to their preset shape projecting radially outwardly from the rest of stent 650. Once engaging arms 670 are in their preset shape, prosthetic heart valve 600 may be pulled (or pushed) toward left atrium 122 until engaging arms 670 hook under native mitral valve leaflets 136, 138, as shown in FIG. 6G. The rounded configuration of strut 670c may reduce the likelihood of trauma to native tissue captured by engaging arms 670. When hooked around native mitral valve leaflets 136, 138, engaging arms 670 help anchor prosthetic heart valve 600 within native valve annulus VA and resist its migration into left atrium 122.

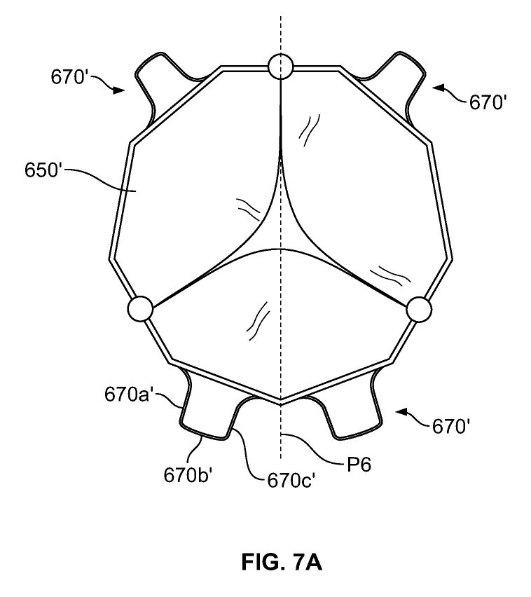

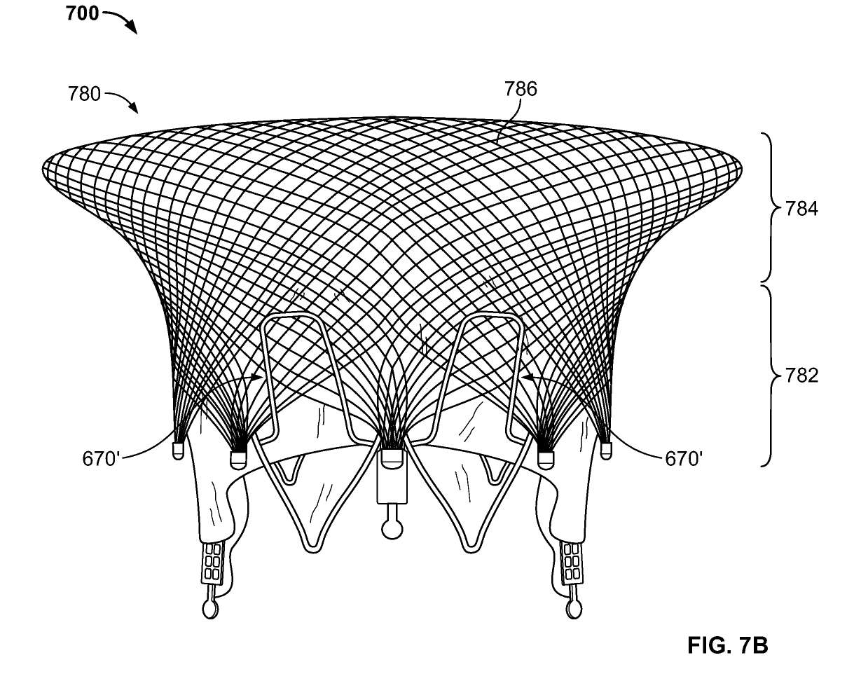

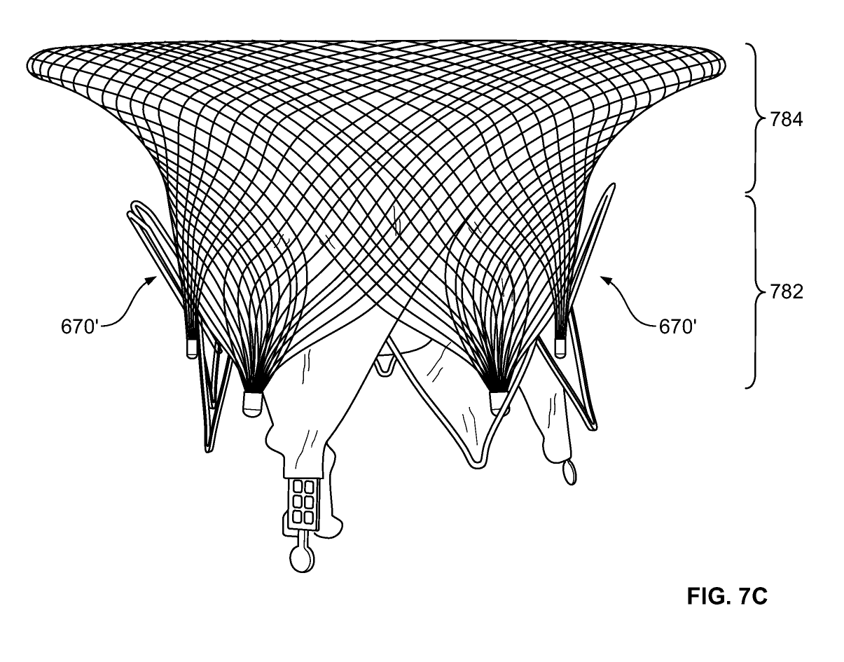

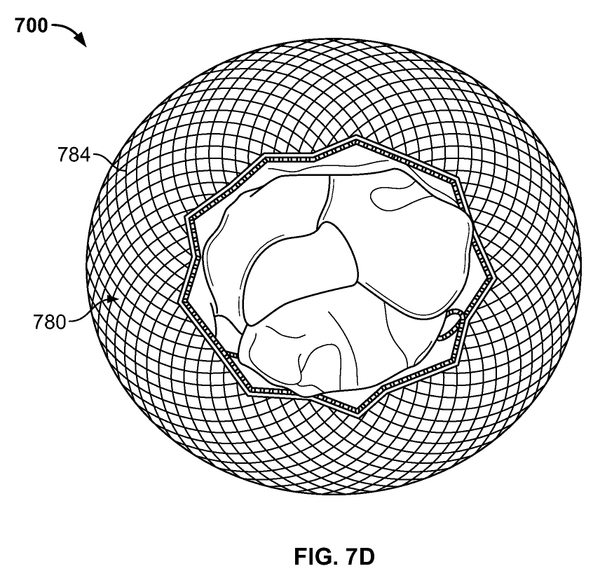

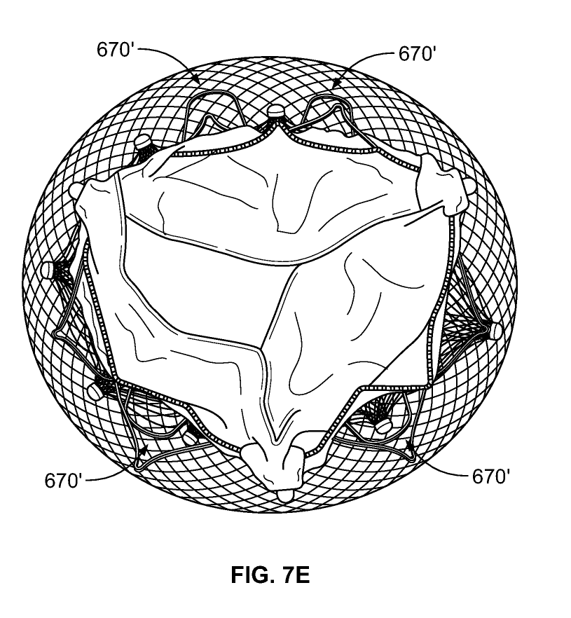

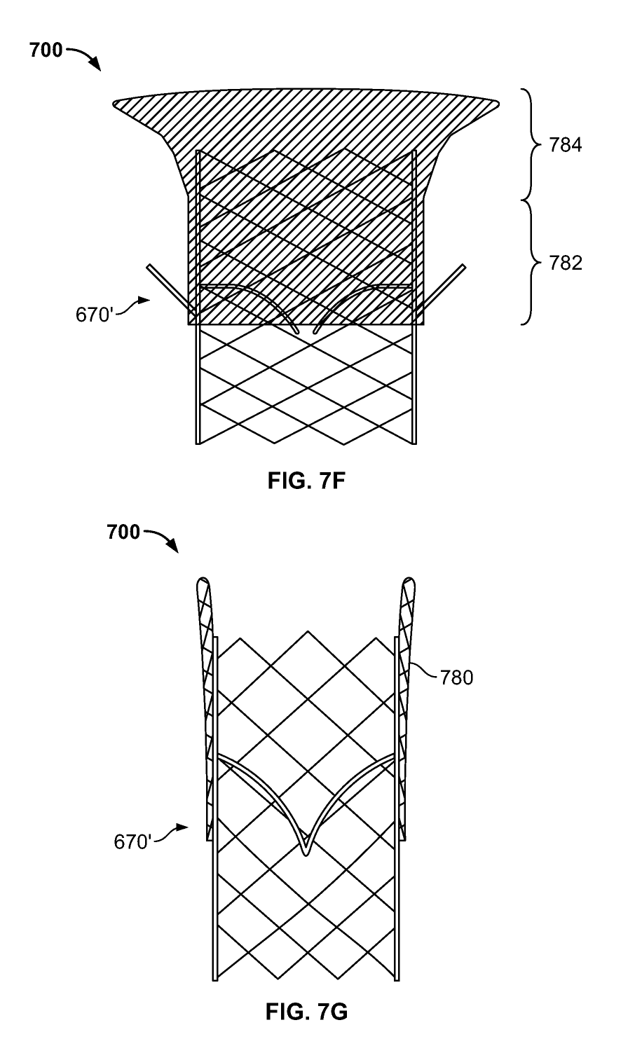

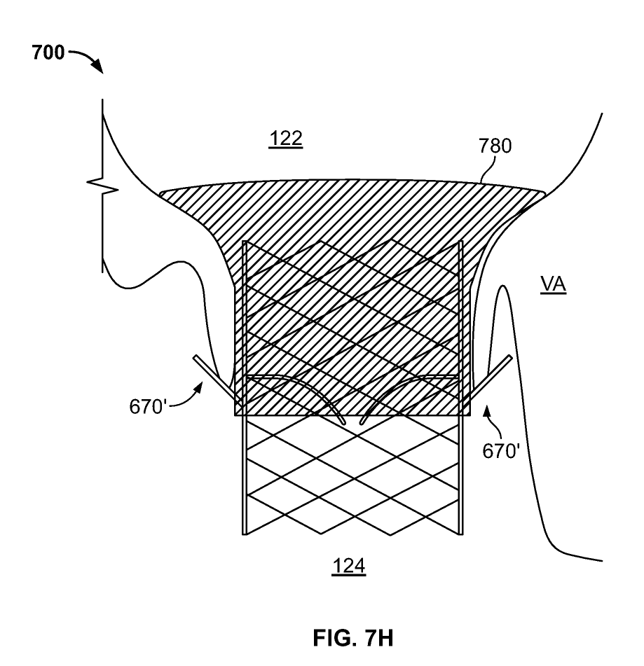

Similar to stent 450, stent 650 of prosthetic heart valve 600 may be formed with an odd number of cells in each circumferential row rather than an even number. Stent 650′, shown in FIG. 7A, is similar to stent 650 with the exception that it has two annular rows of nine cells each. With this configuration, engaging arms 670′ may be situated around the circumference of stent 650′ so that they are symmetric relative to one plane P6. Prosthetic heart valve 700, shown in FIGS. 7B-H, incorporates flange 780 with stent 650′. Flange 780, and its relation to stent 650′, may be similar or identical to the flange 580 of prosthetic heart valve 500 and its relation to stent 450′. For example, flange 780 may include a plurality of braided strands or wires 786 arranged in three dimensional shapes. The body portion 782 and flared portion 784 of flange 780 may also be similar or identical to the corresponding portions of flange 580, with body portion 782 being coupled to stent 650′ by sutures, for example. Similar to prosthetic heart valve 500, the engaging arms 670′ of prosthetic heart valve 700 are shape-set so that, in the absence of applied forces, the body portion 782 of flange 780 is positioned between the struts 670a′-670c′ forming engaging arms 670′ and the remainder of stent 650′. Similarly, prosthetic heart valve 700 may also include a valve assembly having three leaflets attached to a cylindrical cuff in substantially the same manner as described above in connection with prosthetic heart valves 400 and 600.

Prosthetic heart valve 700 may be delivered to the implant site in the collapsed condition, shown in FIG. 7G, and may be transitioned to the expanded condition near native mitral valve 130. Engaging arms 670′ revert to the preset shape in a similar manner as described above in connection with the engaging arms of prosthetic heart valve 600, capturing native mitral valve leaflets 136, 138 between engaging arms 670′ and corresponding portions of stent 650′, as shown in FIG. 7H. Flange 780 also transitions from the collapsed condition to the expanded condition, assuming its preset shape shown in FIG. 7H. Similar to flange 580 of prosthetic heart valve 500, flange 780 of prosthetic heart valve 700 expands to help anchor prosthetic valve 700 within native valve annulus VA. Flange 780 may also include a fabric layer, similar to fabric layer 588, to provide additional sealing against PV leak. As with prosthetic heart valve 500 described above, the combination of engaging arms 670′ and flange 780 securely anchors prosthetic heart valve 700 within native valve annuls VA and limits its migration toward either the left atrium or the left ventricle.

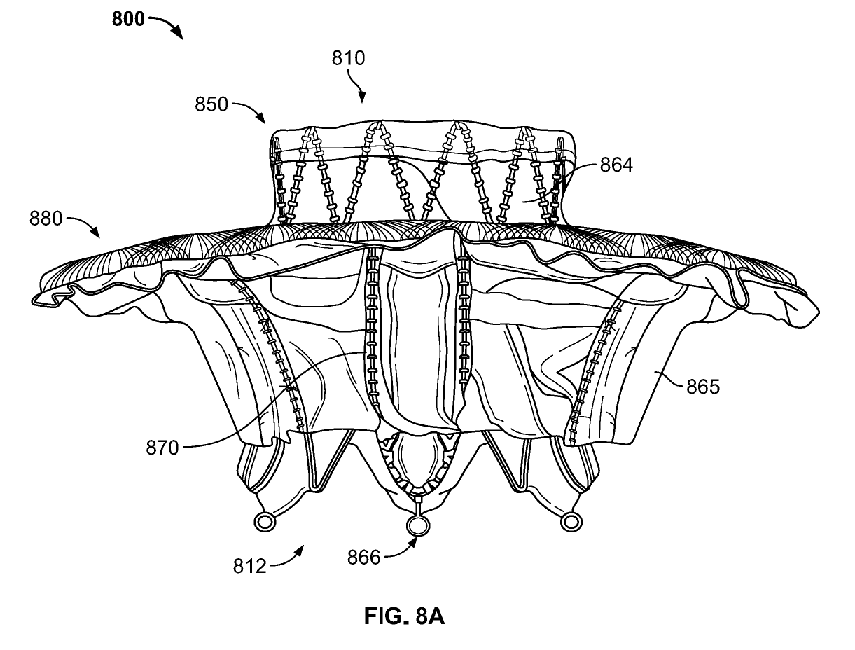

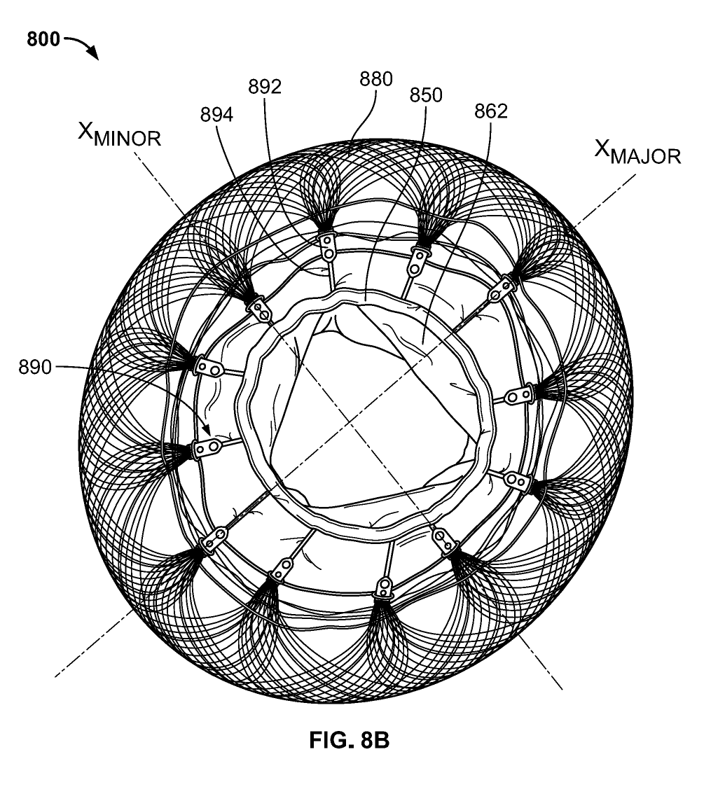

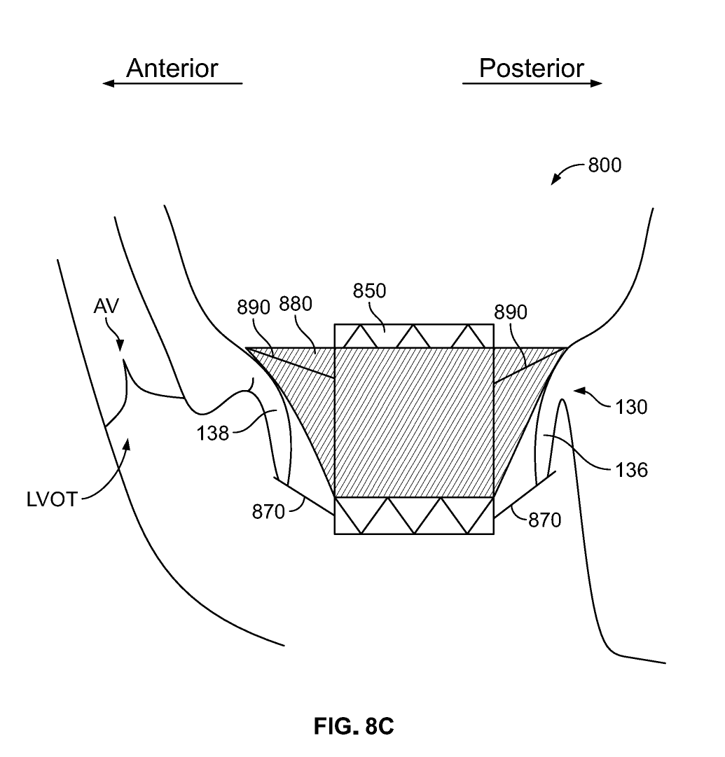

FIG. 8A illustrates a side view of a prosthetic heart valve 800 according to another aspect of the disclosure. Prosthetic heart valve 800 may be substantially similar to prosthetic heart valves 500 and 700 in many respects. For example, prosthetic heart valve 800 may include an inflow end 810, and outflow end 812, and a valve portion 850 that may be substantially similar or identical to prosthetic heart valve 400 or 600. Prosthetic heart valve 800 may include a flange portion 880 that may be substantially similar or identical to flange 580 or flange 780. It should be understood that, in FIG. 8A, valve portion 850 is shown with a cuff 864 attached thereto, which may be similar or identical to other cuffs described above. Further, a second cuff 865, which may be formed of any of the cuff materials described above, may be coupled to an outer periphery of flange portion 880 and may also cover one or more anchor arms or engaging arms 870, which may be substantially similar or identical to other anchor arms or engagement arms described herein, including engagement arms 670. As shown best in FIG. 8A, prosthetic heart valve 800 may include one or more retainers 866 adapted to mate with complementary structures on a delivery device, with the retainers helping to avoid unintentional decoupling of the prosthetic heart valve from the delivery device during deployment at the native valve annulus, such as the native mitral valve.

FIG. 8B is a top view of prosthetic heart valve 800, looking down at the inflow end of the prosthetic heart valve, with the leaflets 862 of the prosthetic heart valve in an open condition. In the illustrated embodiment, valve portion 850 is substantially cylindrical and includes three prosthetic leaflets 862, although more or fewer than three leaflets may be suitable, and shapes other than cylindrical may be suitable. Flange portion 880, on the other hand, is substantially elliptical in the illustrated embodiment, the elliptical shape generally including a major axis X_{MAJOR} and a minor axis X_{MINOR}. Although other shapes of flange portion 880 may be suitable, the native mitral valve 130 is typically elliptical, and thus a corresponding elliptical shape of the flange portion may better correspond with the native anatomy. As illustrated, although valve portion 850 is substantially cylindrical and flange portion 880 is substantially elliptical, the valve portion is substantially centered at the intersection of the major axis X_{MAJOR} and minor axis X_{MINOR} of the elliptical flange portion. In order to obtain this configuration, a variety of different sized connectors 890 may connect flange portion 880 to valve portion 850. Each connector may include a first portion 892 which may be, for example, a crimp tube. One or a group of wires of flange portion 880 may be gathered and coupled to the first portion 892 of connector 890. Each connector 890 may include a second portion 894 extending between a first end coupled to the first portion 892 of the connector and a second end coupled to valve portion 850. The second portion 894 of connector 890 may take the form of a strand, wire, or other substantially straight member, and may be coupled at its second end to a strut or other portion of the stent of valve portion 850. Second portion 894 may be coupled to the stent of valve portion 850 via welding, sutures, adhesives, or any other suitable connection method. In one embodiment, second portion 894 may be integral with the stent of valve portion 850, for example by laser cutting the stent from a single tube, with the second portions of connectors 890 being cut from the same tube. It should be understood that the second portion 894 of connectors 890 may be the only structures coupling flange portion 880 to the stent of valve portion 850. The second portions 894 of connectors 890 may have different lengths depending on their positions around the circumference of valve portion 850. For example, the second portions 894 of connectors 890 positioned along or adjacent major axis X_{MAJOR} may be longer than all other second portions. On the other hand, the second portions 894 of connectors 890 positioned along or adjacent minor axis X_{MINOR} may be shorter than all other second portions. In some embodiments, the second portions 894 of the connectors 890 positioned on or adjacent minor axis X_{MINOR} may be omitted, with the first portions 892 of the connectors being directly coupled to valve portion 850. By varying the lengths of the second portions 894 of connectors 890 so that the second portions are shortest along minor axis X_{MINOR} and increase in length toward major axis X_{MAJOR}, flange portion 880 may maintain an elliptical shape while valve portion 850 may maintain a cylindrical shape and be positioned substantially at the intersection of the major and minor axes.

FIG. 8C shows a highly schematic illustration of prosthetic heart valve 800 positioned within the valve annulus VA of native mitral valve 130. The native aortic valve AV is also illustrated to show the general positional relationship between the native aortic valve and native mitral valve 130, although the drawing is not intended to be to scale. As illustrated in FIG. 8C, one or more engagement arms 870 may engage or hook around the native anterior leaflet 138 and posterior leaflet 136 of native mitral valve 130 to help resist migration of prosthetic heart valve 800 into the left atrium. Similarly, a top or flared portion of flange portion 880 may contact the native mitral valve annulus facing the left atrium to help resist migration of the prosthetic heart valve 800 into the left ventricle. It should be understood that, although the exterior perimeter of valve portion 850 may be spaced a distance from the interior surface of flange portion 880, cuff 864 and/or second cuff 865 may help ensure that blood does not flow from the left atrium to the left ventricle (or vice versa) through the space between the valve portion and the flange portion. For example, second cuff 865 may be positioned on an exterior and/or interior surface of flange portion 880 and may extend so that it couples to valve portion 850 in order to help ensure that blood is only able to flow through the prosthetic valve 800 via the space between leaflets 862 when the leaflets are in an open condition, as best seen in FIG. 8B.

Referring again to FIG. 8C, although prosthetic heart valve 800 may be effective at allowing blood to flow in the antegrade direction through the valve portion 850 and restricting blood from flowing in the retrograde direction in a manner similar to a properly functioning native mitral valve, potential drawbacks may be present depending on the particular geometry and placement of prosthetic heart valve 800 in the heart. For example, as noted above, if the outflow end of one or both of valve portion 850 and flared portion 880 extend a large distance into the left ventricle, the structure may obstruct blood flowing along the LVOT through native aortic valve AV. In addition, depending on the size of prosthetic heart valve 800 and whether it is angled with respect to the longitudinal axis of native mitral valve 130 when implanted, portions of the prosthetic heart valve may contact the septum separating the left and right ventricles and interfere with electrical conduction in the tissue, which may result in heart pacing irregularities. One way to reduce the likelihood of either of these problems arising is to shift the valve portion posteriorly, as described below.

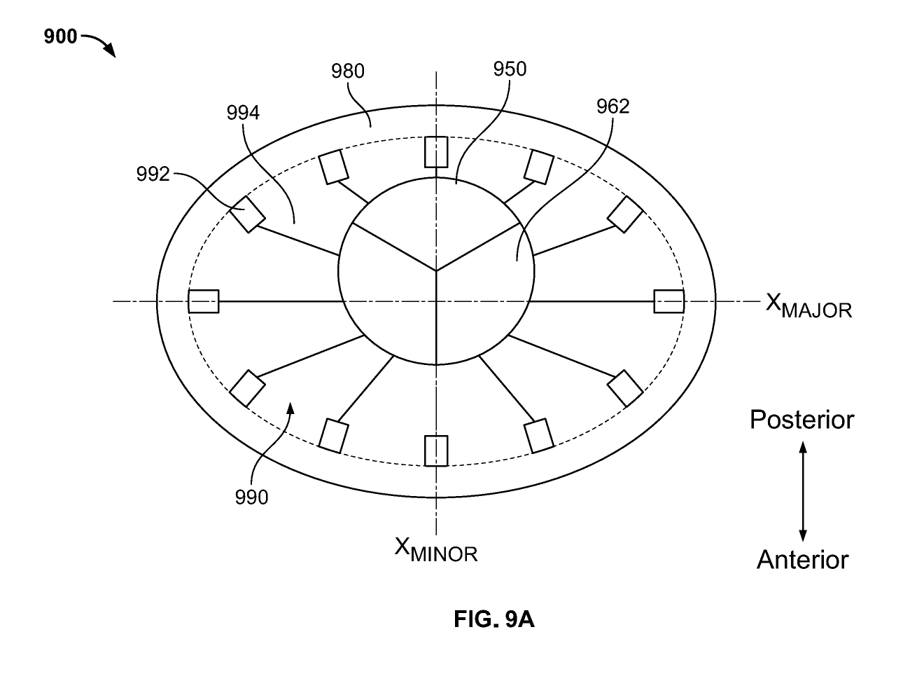

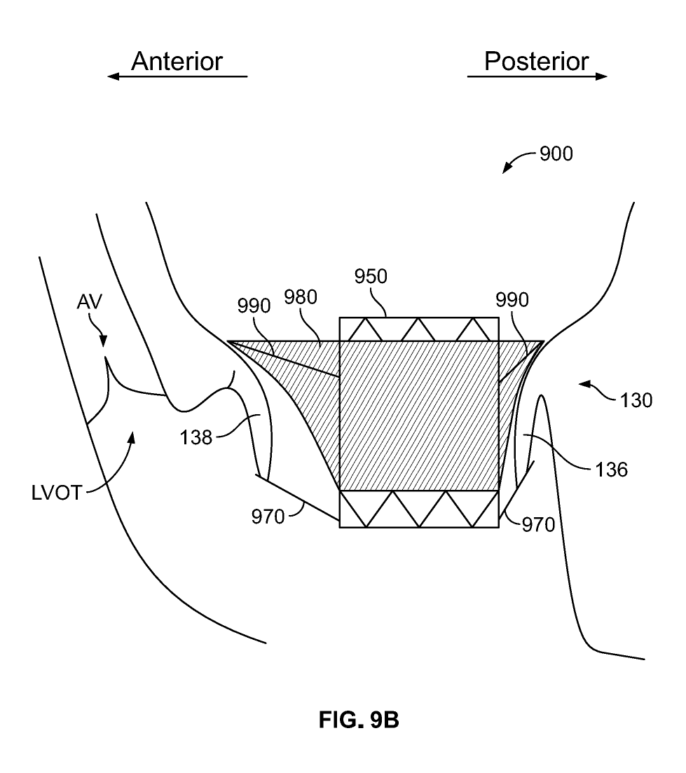

FIG. 9A illustrates a top view of prosthetic heart valve 900 looking toward the inflow end thereof. Prosthetic heart valve 900 may be substantially similar to prosthetic heart valve 800, with the main difference being the position of the valve portion relative to the flange portion, and the configuration of the connectors that dictate this relative positioning. Similar to prosthetic valve 800, prosthetic valve 900 may include a substantially elliptical flange portion 980 having a major axis X_{MAJOR} and a minor axis X_{MINOR}. Also similar to prosthetic valve 800, prosthetic valve 900 may include a substantially cylindrical valve portion 950 with three prosthetic leaflets 962. However, while valve portion 950 is preferably centered in the direction of the major axis X_{MAJOR} of flange portion 980, the valve portion is preferably offset in the posterior direction from the major axis of the flange portion. In other words, whereas the central longitudinal axis of valve portion 850 is substantially coaxial with the central longitudinal axis of flange portion 880, the central longitudinal axis of valve portion 950 is posteriorly offset from the central longitudinal axis of flange portion 980. Thus, when implanted, while the central longitudinal axis of valve portion 850 is substantially aligned with the central longitudinal axis of native mitral valve 130 as shown in FIG. 8C, the central longitudinal axis of valve portion 950 when implanted is posteriorly offset from the central longitudinal axis of the native mitral valve, as shown in FIG. 9B. Preferably, the central longitudinal axis of valve portion 950 is substantially parallel to the central longitudinal axis of flange portion 980. In one embodiment, the central longitudinal axis of valve portion 950 is offset from the central longitudinal axis of flange portion 980 (and/or the central longitudinal axis of native mitral valve 130 after implantation) by a distance of between about 4 mm and about 8 mm in the posterior direction. In some embodiments, that posterior offset may be between about 5 mm and about 7 mm. In another embodiment, that posterior offset may be about 6 mm.

Still referring to FIG. 9A, one way to provide the offset of valve portion 950 relative to flange portion 980 is to modify connectors 990 compared to connectors 890. The general structure of connectors 990 may be substantially similar or identical to that of connectors 890. For example, each connector 990 may include a first portion 992 which may connect to one or a group of wires of flange portion 980. In one embodiment, first portion 992 that be a crimp tube that is crimped over one or a group of wires of flange portion 980. Each connector 990 also may include a second portion 994 that may take the form of a strand, wire, or other substantially straight member, and may be coupled at a first end to first portion 992 and at a second end to a strut or other stent portion of valve portion 950. The second portions 994 of connectors 990 may have different lengths in order to help position valve portion 950 posterior to the major axis X_{MAJOR}. For example, the second portion 994 of one connector 990 extending along the minor axis X_{MINOR} on the anterior side of prosthetic valve 900 may have a large length compared to the second portion of another connector extending along the minor axis on the posterior side of the prosthetic valve.

In one embodiment, the second end of the second portion 994 of each connector 990 may be positioned at substantially the same location in the inflow-to-outflow direction of the valve portion 950. In such configuration, the connectors 990 positioned near the posterior side of prosthetic heart valve 900 would extend at a greater angle relative the central longitudinal axis of the prosthetic valve compared to connectors positioned near the anterior side of the prosthetic heart valve, as shown in FIG. 9B. In other words, anterior connectors 990 may be closer to being orthogonal to the longitudinal axis of the prosthetic heart valve 900 than are the posterior connectors.

Although not separately labeled, prosthetic heart valve 900 may include a first cuff on the valve portion 950 that is substantially similar or substantially identical to cuff 864 on valve portion 850, and a second cuff on the flange portion 980 may be substantially similar or substantially identical to second cuff 865 on flange portion 880, so that blood does not pass through prosthetic heart valve 900 other than past leaflets 962 when they are in the open condition. Further, prosthetic heart valve 900 may include engagement arms 970 that are substantially similar in most respects to engagement arms 870. However, because valve portion 950 has a posterior offset, an anterior engagement arm 970 may be longer than a posterior engagement arm, as the native anterior leaflet 138 may be a greater distance from the valve portion 950 than is the native posterior leaflet 136 when implanted.

Although prosthetic heart valves 800, 900 are both described as having first and second cuffs on valve portions 850, 950 and flange portions 880, 980, respectively, that help ensure blood flows only past leaflets 862, 962 in the open condition, additional cuffs may be provided. As described and illustrated, when prosthetic heart valves 800, 900 are implanted, blood may flow from the left atrium into the space between the exterior surfaces of valve portions 850, 950 and the interior surfaces of flange portions 880, 980, although the blood cannot flow from that position into the left ventricle due to the presence of the first and/or second cuffs. In other embodiments, it may be preferable to include a substantially annular cuff or sealing member extending from an exterior circumference of the valve portion to the inflow edge of the flange portion. With such an annular cuff, blood may be prevented from entering the space between the exterior surfaces of valve portions 850, 950 and the interior surfaces of flange portions 880, 980 in the first place.

In an exemplary method of use, prosthetic heart valve 900 may be transitioned into a collapsed condition similar to that shown for valve 700 in FIG. 7G, and loaded into a delivery device, an outer sheath of the delivery device covering the prosthetic heart valve and maintaining it in the collapsed condition. The collapsed prosthetic heart valve 900 may be passed through the patient's body (for example, through the atrial septum using a transseptal (TS) approach or through the left ventricle using a transapical (TA) approach) and positioned adjacent native mitral valve 130. The outer sheath may be translated to allow prosthetic valve 900 to expand into the expanded condition, with flange portion 980 in contact with an atrial side of the native mitral valve annulus and valve portion 950 positioned between native anterior leaflet 138 and native posterior leaflet 136. If engagement arms 970 are included, they may transition to a pre-set shape, such as that shown in FIG. 9B, to hook over or otherwise engage native leaflets 136, 138. As noted above, upon implantation, the central longitudinal axis of valve portion 950 is offset from, and preferably parallel to, the central longitudinal axis of native mitral valve 130.

According to one embodiment of the disclosure, a collapsible and expandable prosthetic mitral valve comprises:

a stent having an inflow end, an outflow end, and a first central longitudinal axis extending from the inflow end to the outflow end in an expanded condition of the prosthetic mitral valve;

a valve assembly disposed within the stent; and

a flange formed of a braided mesh and having a body portion coupled to the stent and a flared portion adjacent the inflow end of the stent, a second central longitudinal axis extending through the flared portion in the expanded condition of the prosthetic mitral valve,

wherein the first central longitudinal axis is offset from the second central longitudinal axis; and/or

the first central longitudinal axis is parallel to the second central longitudinal axis; and/or

the flared portion is substantially elliptical in the expanded condition of the prosthetic mitral valve and includes a major axis and a minor axis; and/or

the first central longitudinal axis is positioned on the minor axis and is offset from the major axis; and/or

a plurality of connectors coupling the flared portion of the flange to the stent; and/or

the flared portion of the flange includes an anterior portion on a first side of the major axis and a posterior portion on a second side of the major axis, the first central longitudinal axis being positioned on the second side of the major axis; and/or

a first one of the connectors couples the anterior portion of the flange to an anterior portion of the stent and has a first length, and a second one of the connectors couples the posterior portion of the flange to a posterior portion of the stent and has a second length, the first length being greater than the second length; and/or

the stent is substantially cylindrical in the expanded condition of the prosthetic mitral valve; and/or

an anterior engagement arm and a posterior engagement arm each having a first end pivotably coupled to the stent and a free end extending toward the inflow end of the stent; and/or

the anterior engagement arm is longer than the posterior engagement arm.

In another embodiment of the disclosure, a method of implanting a prosthetic mitral valve comprises:

introducing a delivery device to a native mitral valve annulus while the prosthetic mitral valve is maintained in a collapsed condition by the delivery device;

transitioning the prosthetic mitral valve into an expanded condition so that a stent of the prosthetic mitral valve is positioned within the native mitral valve annulus to implant the prosthetic mitral valve, the stent including a valve assembly disposed therein, and so that a flared portion of a flange of the prosthetic mitral valve contacts an atrial side of the native mitral valve annulus, the flange being formed of a braided mesh and having a body portion coupled to the stent,

wherein upon implantation of the prosthetic mitral valve, the native mitral valve annulus has a first central longitudinal axis, and the stent has a second central longitudinal axis offset from the first central longitudinal axis; and/or

upon implantation of the prosthetic mitral valve, the second central longitudinal axis is positioned closer to a posterior leaflet of the native mitral valve than to an anterior leaflet of the native mitral valve; and/or

upon implantation of the prosthetic mitral valve, the first central longitudinal axis is parallel to the second central longitudinal axis; and/or

in the expanded condition of the prosthetic mitral valve, the flared portion is substantially elliptical and includes a major axis and a minor axis; and/or

the second central longitudinal axis is positioned on the minor axis and is offset from the major axis; and/or

a plurality of connectors couple the flared portion of the flange to the stent; and/or upon implantation of the prosthetic mitral valve, a first one of the connectors is positioned nearer an anterior leaflet of the native mitral valve than is a second one of the connectors, the first one of the connectors having a length greater than a length of the second one of the connectors; and/or

the stent is substantially cylindrical in the expanded condition of the prosthetic mitral valve; and/or

engaging a free end of an anterior engagement arm of the stent with an anterior leaflet of the native mitral valve and engaging a free end of a posterior engagement arm of the stent with a posterior leaflet of the native mitral valve, each engagement arm having a first end pivotably coupled to the stent; and/or

the anterior engagement arm has a first length from the first end to the free end and the posterior engagement arm has a second length form the first end of the free end that is less than the first length.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, features of one embodiment of the invention may be combined with features of one or more other embodiments of the invention without departing from the scope of the invention.

1. A collapsible and expandable prosthetic mitral valve, comprising:
a stent having an inflow end, an outflow end, and a first central longitudinal axis extending from the inflow end to the outflow end in an expanded condition of the prosthetic mitral valve;
a valve assembly disposed within the stent; and
a flange formed of a braided mesh and having a body portion coupled to the stent and a flared portion adjacent the inflow end of the stent, a second central longitudinal axis extending through the flared portion in the expanded condition of the prosthetic mitral valve,
wherein the first central longitudinal axis is offset from the second central longitudinal axis.

2. The collapsible and expandable prosthetic mitral valve of claim 1, wherein the first central longitudinal axis is parallel to the second central longitudinal axis.

3. The collapsible and expandable prosthetic mitral valve of claim 1, wherein the flared portion is substantially elliptical in the expanded condition of the prosthetic mitral valve and includes a major axis and a minor axis.

4. The collapsible and expandable prosthetic mitral valve of claim 3, wherein the first central longitudinal axis is positioned on the minor axis and is offset from the major axis.

5. The collapsible and expandable prosthetic mitral valve of claim 4, further comprising a plurality of connectors coupling the flared portion of the flange to the stent.

6. The collapsible and expandable prosthetic mitral valve of claim 5, wherein the flared portion of the flange includes an anterior portion on a first side of the major axis and a posterior portion on a second side of the major axis, the first central longitudinal axis being positioned on the second side of the major axis.

7. The collapsible and expandable prosthetic mitral valve of claim 6, wherein a first one of the connectors couples the anterior portion of the flange to an anterior portion of the stent and has a first length, and a second one of the connectors couples the posterior portion of the flange to a posterior portion of the stent and has a second length, the first length being greater than the second length.

8. The collapsible and expandable prosthetic mitral valve of claim 1, wherein the stent is substantially cylindrical in the expanded condition of the prosthetic mitral valve.

9. The collapsible and expandable prosthetic mitral valve of claim 1, further comprising an anterior engagement arm and a posterior engagement arm each having a first end pivotably coupled to the stent and a free end extending toward the inflow end of the stent.

10. The collapsible and expandable prosthetic mitral valve of claim 9, wherein the anterior engagement arm is longer than the posterior engagement arm.

11. A method of implanting a prosthetic mitral valve, the method comprising:
introducing a delivery device to a native mitral valve annulus while the prosthetic mitral valve is maintained in a collapsed condition by the delivery device;
transitioning the prosthetic mitral valve into an expanded condition so that a stent of the prosthetic mitral valve is positioned within the native mitral valve annulus to implant the prosthetic mitral valve, the stent including a valve assembly disposed therein, and so that a flared portion of a flange of the prosthetic mitral valve contacts an atrial side of the native mitral valve annulus, the flange being formed of a braided mesh and having a body portion coupled to the stent,
wherein upon implantation of the prosthetic mitral valve, the native mitral valve annulus has a first central longitudinal axis, and the stent has a second central longitudinal axis offset from the first central longitudinal axis.

12. The method of claim 11, wherein upon implantation of the prosthetic mitral valve, the second central longitudinal axis is positioned closer to a posterior leaflet of the native mitral valve than to an anterior leaflet of the native mitral valve.

13. The method of claim 11, wherein upon implantation of the prosthetic mitral valve, the first central longitudinal axis is parallel to the second central longitudinal axis.

14. The method of claim 11, wherein in the expanded condition of the prosthetic mitral valve, the flared portion is substantially elliptical and includes a major axis and a minor axis.

15. The method of claim 14, wherein the second central longitudinal axis is positioned on the minor axis and is offset from the major axis.

16. The method of claim 15, wherein a plurality of connectors couple the flared portion of the flange to the stent.

17. The method of claim 16, wherein upon implantation of the prosthetic mitral valve, a first one of the connectors is positioned nearer an anterior leaflet of the native mitral valve than is a second one of the connectors, the first one of the connectors having a length greater than a length of the second one of the connectors.

18. The method of claim 11, wherein the stent is substantially cylindrical in the expanded condition of the prosthetic mitral valve.

19. The method of claim 11, further comprising engaging a free end of an anterior engagement arm of the stent with an anterior leaflet of the native mitral valve and engaging a free end of a posterior engagement arm of the stent with a posterior leaflet of the native mitral valve, each engagement arm having a first end pivotably coupled to the stent.

20. The method of claim 19, wherein the anterior engagement arm has a first length from the first end to the free end and the posterior engagement arm has a second length form the first end of the free end that is less than the first length.
